(19) United States
(12) Patent Application Publication
(10) Pub. No.: US 2018/0078120 A1
(43) Pub. Date: Mar. 22, 2018

(54) ENDOSCOPE HAVING INTEGRATED VISUAL FIELD ENHANCEMENT SYSTEM

(71) Applicant: FLOSHIELD, INC., Cupertino, CA (US)

(72) Inventors: Wayne L. POLL, New Albany, OH (US); Gregory P. DRACH, Liberty Township, OH (US)

(21) Appl. No.: 15/566,503

(22) PCT Filed: Apr. 15, 2016
(86) PCT No.: PCT/US16/27986

§ 371 Date: Oct. 13, 2017

Related U.S. Application Data
(60) Provisional application No. 62/148,438, filed on Apr. 16, 2015.
(60) Provisional application No. 62/164,163, filed on May 20, 2015.

(51) Int. Cl. A61B 1/00; A61B 1/015; A61B 1/12; A61B 1/005; A61B 1/05; A61B 1/273; A61B 1/012; A61B 1/31; A61B 1/303; A61B 1/267; A61B 1/227; A61B 1/233; A61B 1/24; A61M 13/00; A61B 1/313; A61B 1/018

(52) U.S. Cl. CPC A61B 1/00091; A61B 1/018; A61B 1/126; A61B 1/00101; A61B 1/00105; A61B 1/00078; A61B 1/0052; A61B 1/00096; A61B 1/00128; A61B 1/00163; A61B 1/05; A61B 1/00114; A61B 1/121; A61B 1/00103; A61B 1/2736; A61B 1/0125; A61B 1/31; A61B 1/303; A61B 1/2676; A61B 1/227; A61B 1/233; A61B 1/24; A61B 1/00135; A61B 1/0014; A61B 1/051; A61M 13/003; A61B 1/3132; A61B 1/015

(57) ABSTRACT

A scope includes an elongate body, a lens at the distal end of the elongate body, at least one conduit, and a view optimizing assembly. The conduit is configured to connect to an air supply. The view optimizing assembly extends from the distal end of the elongate body past the lens and includes a first lumen and a second lumen, a plurality of dividers separating the lumens, and a deflector. The first and second lumens are in fluid communication with the conduit and are sized such that a single velocity flow from the conduit separates into a first flow through the first lumen and a second flow through the second lumen. The first flow has a higher velocity than the second flow. The deflector assembly is configured such that air exiting the first and second lumens combines to keep debris off of the lens.

### CROSS-REFERENCE TO RELATED APPLICATIONS

This application claims priority to U.S. Provisional Patent Application No. 62/148,438, filed Apr. 16, 2015, titled “ENDOSCOPE HAVING INTEGRATED VISUAL FIELD ENHANCEMENT SYSTEM,” and to U.S. Provisional Patent Application No. 62/164,163, filed May 20, 2015, and titled “NON-ROUND ENDOSCOPE WITH SHEATH HAVING INTEGRATED VISUAL FIELD ENHANCEMENT SYSTEM,” each of which is incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described here are a number of different endoscope, sheath, and endoscope tip cap configurations having one or more visual field improvement mechanisms, such as defogging, particle removal, or clearance.

### BACKGROUND

An endoscope is a medical instrument having an elongate body that may take on a number of different form factors depending on the type of medical procedure being performed with the endoscope. An endoscope elongate body is generally categorized as being rigid, semi-rigid or flexible. Semi-rigid and flexible scopes typically include some form of steering or bending mechanism. Most importantly, an endoscope will include a lighting system and some visualization component to provide imaging information of the area at the distal end of the endoscope in the field of view of the visualization component.

Oftentimes the quality of the surgical field image provided by the endoscope visualization system is impaired either because of fogging caused by the environment (i.e., moist and humid) or because of by-products of a surgical procedure (e.g., tissue, blood, smoke) obstructing the view. Accordingly, improvements to endoscopes are needed that ensure that the visual field remains clear during use in a surgical procedure.

### SUMMARY OF THE DISCLOSURE

In general, in one embodiment, a scope includes an elongate body having a proximal end and a distal end, a lens at the distal end of the elongate body, at least one conduit extending from the proximal end to the distal end configured to connect to an air supply, and a view optimizing assembly extending from the distal end of the elongate body past the lens. The view optimizing assembly includes a first lumen and a second lumen, a plurality of dividers separating the lumens, and a deflector assembly configured such that air exiting the first and second lumens combines to keep debris off of the lens. The first and second lumens are in fluid communication with the at least one conduit and are configured such that a single velocity flow from the at least one conduit separates into a first flow through the first lumen and a second flow through the second lumen, the first flow having a higher velocity than the second flow.

This and other embodiments can include one or more of the following features. The at least one conduit can extend within the elongate body. The scope can further include a sheath extending around the elongate body. The at least one conduit can extend between an outer circumference of the elongate body and an inner circumference of the sheath. The at least one conduit can extend within the sheath. The at least one conduit can include a plurality of conduits. The deflector assembly can further include a plenum section configured to allow air from the plurality of conduits to combine into a single velocity air flow before entering the first and second lumens. The plurality of dividers can include a plurality of stand-offs configured to touch a surface of the lens. The at least one conduit can include a plurality of conduits, and the stand-offs can extend from a wall between the conduits. The at least one conduit can include a single conduit, and the stand-offs can divide the air into the first and second lumens. The deflector, the distal end of the elongate body, and the dividers together can form a first nozzle in communication with the first lumen and a second nozzle in communication with the second lumen. A length of each lumen can be between 0.005 inches and 0.010 inches. The air exiting the first and second lumens can combine to form a vortex to keep debris off of the lens. The elongate body can be flexible. The elongate body can be rigid. The view optimizing assembly can be attached to the elongate body with a locking mechanism. The view optimizing assembly can be integral with the elongate body. The first lumen can be larger than the second lumen such that the first flow has a higher velocity than the second flow.

In general, in one embodiment, a view optimizing assembly for a scope includes an elongate body configured to extend from a distal end of a scope past a lens of the scope, a first lumen and a second lumen within the elongate body, a plurality of dividers separating the lumens, and a deflector assembly configured such that air exiting the first and second lumens combines to keep debris off of the lens. The first and second lumens are in fluid communication with at least one conduit of a scope and are configured such that a single velocity flow from the at least one conduit separates into a first flow through the first lumen and a second flow through the second lumen, the first flow having a higher velocity than the second flow.

This and other embodiments can include one or more of the following features. The at least one conduit can include a plurality of conduits. The deflector assembly can further include a plenum section configured to allow air from the plurality of conduits to combine into a single velocity air flow before entering the first and second lumens. The plurality of dividers can include a plurality of stand-offs configured to touch a surface of the lens. A length of each lumen can be between 0.005 inches and 0.010 inches. The air exiting the first and second lumens can combine to form a vortex to keep debris off of the lens. The view optimizing assembly can be configured to attach to the scope with a locking mechanism. The first lumen can be larger than the second lumen such that the first flow has a higher velocity than the second flow.

In general, in one embodiment, a scope includes an elongate body having a proximal end and a distal end, an interior lumen within the elongate body extending from the proximal end to the distal end, a tip face, a gas conduit within the elongate body lumen, a visualization component in the tip face, and a tip cap. A distal end includes a tip engagement region. A tip face is adjacent to the tip engagement region and covering the interior lumen. A gas conduit within the elongate body lumen has an outlet in the tip face and an inlet at the proximal end of the elongate body. A tip cap is configured to releasably couple with the tip engagement region. The tip cap includes an opening sized for use with the visualization component and at least one stand-off. When the tip cap is coupled to the tip engagement region, the opening is positioned around the visualization component and the one or more stand offs engage a portion of the tip face such that a gas flow from the outlet is directed towards the opening to improve viewing through the visualization component.

This and other embodiments can include one or more of the following features. The scope can be configured such that viewing through the visualization component is improved by one or more of: a gas flow pattern relative to the visualization component to remove condensation therefrom, a gas flow pattern relative to the visualization component to form an air barrier to reduce or minimize particles in the visual field of the visualization component and a gas flow pattern relative to the visualization component to facilitate removal of a fluid applied to the visualization component. The scope can further include a visualization component cable connected to the visualization component and in communication with the proximal end of the elongate body. An overall dimension, such as a diameter, of the tip engagement region can be less than the overall dimension, such as a diameter, of the elongate body proximal portion. An overall dimension of the tip engagement region when coupled to the tip cap can be more than the overall dimension of an elongate body proximal portion. An overall dimension of the tip engagement region when coupled to the tip cap can be about the same as an overall dimension of an elongate body proximal portion. The tip cap can be configured to releasably couple with the tip engagement region using a complementary pair of elastic snap fit features. The tip cap can be configured to releasably couple with the tip engagement region using a threaded connection. The scope can further include a handle on the elongate body proximal end supporting the gas conduit inlet and a visualization component cable. The scope can further include a liquid conduit within the elongate body lumen having a liquid outlet in the tip face and an inlet at the proximal end of the elongate body. The tip cap can further include one or more liquid stand offs positioned such that, when the tip cap is coupled to the tip engagement region, the one or more liquid stand offs are configured to engage a portion of the tip face such that a liquid flow from the liquid outlet is directed towards the opening to further improve viewing through the visualization component. The scope can further include a handle on the elongate body proximal end supporting the gas conduit inlet, the liquid conduit inlet, and a visualization component cable. The elongate body can be rigid, semi-rigid or flexible. The elongate body can be flexible or semi-rigid, and in the scope can further include a handle including a steering mechanism for controlling a bend angle of the elongate body.

In general, a scope includes an elongate body having a proximal end and a distal end, an interior lumen within the elongate body extending from the proximal end to the distal end, a tip face, a first gas conduit and a second gas conduit, a visualization component in the tip face, and a tip cap. The distal end includes a tip engagement region. A tip face is adjacent to the tip engagement region and covering the interior lumen distal end. A tip cap is configured to releasably couple with the tip engagement region. The tip cap includes an opening sized for use with the visualization component and at least one stand-off. When the tip cap is coupled to the tip engagement region, the opening is around the visualization component and the one or more stand offs are engaged with a portion of the tip face such that the gas flows from the first and second gas conduits towards the opening to improve viewing through the visualization component.

This and other embodiments can include one or more of the following features. The first and second gas conduits can be within the elongate body. The scope can further include a gas inlet and a manifold. The gas inlet can be in communication with the manifold, and the manifold can be in communication with the first and second gas conduits. The scope can be configured such that viewing through the visualization component is improved by one or more of: a gas flow pattern relative to the visualization component to remove condensation therefrom, a gas flow pattern relative to the visualization component to form an air barrier to reduce or minimize particles in the visual field of the visualization component and a gas flow pattern relative to the visualization component to facilitate removal of a fluid applied to the visualization component. The scope can further include a visualization component cable connected to the visualization component and in communication with the proximal end of the elongate body. An overall dimension, such as a diameter, of the tip engagement region can be less than an overall dimension, such as a diameter of a proximal portion of the elongate body. An overall dimension of the tip engagement region when coupled to the tip cap can be more than an overall dimension of an elongate body proximal portion. An overall dimension of the tip engagement region when coupled to the tip cap can be about the same as an overall dimension of the elongate body proximal portion. The tip cap can be configured to releasably couple with the tip engagement region using a complementary pair of elastic snap fit features. The tip cap can be configured to releasably couple with the tip engagement region using a threaded connection. The scope can further include a handle on the elongate body proximal end supporting the first and the second gas conduits and a visualization component cable. The scope can further include a liquid conduit within the elongate body lumen having a liquid outlet in the tip face and an inlet at the proximal end of the elongate body. The tip cap can further include one or more liquid stand offs such that, when the tip cap is coupled to the tip engagement region, the one or more liquid stand offs are configured to engage a portion of the tip face such that a liquid flow from the liquid outlet is directed towards the opening to further improve viewing through the visualization component. The elongate body can be rigid. The elongate body can be semi-rigid. The elongate body can be flexible. The scope can have an elongate body that can be flexible or semi-rigid. The scope can further include a handle including a steering mechanism for controlling a bend angle in the elongate body.

In general, in one embodiment, a surgical scope includes an elongate body having a proximal end and a distal end, an interior lumen within the elongate body extending from the proximal end to the distal end, a recessed portion at the elongate body distal end, a tip face directly adjacent to the recessed portion, two or more gas conduits within the elongate body lumen, a gas inlet at the proximal end of the elongate body, a visualization component in the tip face, and a visualization component cable connected to the visualization component and in communication with the proximal end of the elongate body. A recessed portion at the elongate body distal end is configured to releasably couple to a tip cap. A tip face directly adjacent to the recessed portion covers the interior lumen distal end. Each of said two or more gas conduits have an outlet in the tip face and an inlet at a gas manifold. A gas inlet at the proximal end of the elongate body is in communication with the gas manifold. An overall dimension, such as a diameter, of the recessed portion of the elongate body distal end is less than the overall dimension, such as a diameter, of the elongate body proximal portion.

This and other embodiments can include one or more of the following features. The surgical scope can further include a handle on the elongate body proximal end supporting the gas conduit inlet and the visualization component cable. The surgical scope can further include a liquid conduit within the elongate body lumen having an outlet in the tip face and an inlet at the proximal end of the elongate body. The surgical scope can further include a handle on the elongate body proximal end supporting the gas conduit inlet, the liquid conduit inlet and the visualization component cable. The gas manifold can be disposed within the handle. The elongate body can be rigid, semi-rigid or flexible. The scope can have an elongate body that can be flexible or semi-rigid. The handle can further include a steering mechanism for controlling a bend angle in a portion of the flexible or semi-rigid elongate body.

In general, in one embodiment, a scope includes an elongate body having a proximal end and a distal end and a non-round cross section, a visualization component at the elongate body distal end, and an attachment mechanism on the elongate body configured for attachment to a sheath such that, when a sheath is placed around the elongate body and attached thereto with the attachment mechanism, at least one conduit is configured to attach to an air supply and extends from the proximal end to the distal end between an outer circumference of the elongate body and an inner circumference of the sheath.

This and other embodiments can include one or more of the following features. The attachment mechanism can be on a proximal portion of the elongate body and can be configured for sealing engagement with the sheath. The sheath can include a sidewall with an exterior wall having a circular cross section shape and an interior wall configured for complementary engagement with the non-round cross section of the elongate body. The at least one conduit can include a plurality of conduits. The plurality of conduits can be configured to direct air over the visualization component in a vortex. A fluid flow through the conduits can be apportioned so as to adjust the flow characteristics of the fluid discharged from the plurality of conduits relative to the visualization component. When the sheath is placed around the elongate body and attached thereto with the attachment mechanism, the at least one conduit can be connected to a gas nozzle at the distal portion of the conduit. The gas nozzle can be configured to direct air across the visualization component to provide at least one visual field improvement action. When the sheath is placed around the elongate body and attached thereto with the attachment mechanism, one or more stand offs in a distal portion of the sheath can engage with a portion of the elongate body distal end. When the sheath is placed around the elongate body and attached thereto with the attachment mechanism, one or more stand offs in a distal portion of the sheath can engage with a portion of the elongate body distal end and at least two conduits are formed along the elongate body in communication with a sheath gas inlet. A fluid flowing through the sheath gas inlet can pass through the at least two conduits and exit adjacent to the visualization component via one or more openings bounded at least in part by a portion of one or more stand offs and a portion of the elongate body distal end. When the sheath is placed around the elongate body and attached thereto with the attachment mechanism, a distal portion of the sheath having one or more stand offs can be configured to engage a portion of the elongate body distal portion such that a gas flow introduced into the conduit is directed towards the visualization component. When the sheath is placed around the elongate body and attached thereto with the attachment mechanism, a distal portion of the sheath having one or more stand offs can engage a portion of the elongate body distal portion such that a gas flow introduced into the conduit provides at least one visual field improvement action. When the sheath is placed around the elongate body and attached thereto with the attachment mechanism, one or more stand offs in a distal portion of the sheath can engage with a portion of the elongate body distal end and at least two conduits are formed along the elongate body in communication with a sheath gas inlet. A fluid flowing through the sheath gas inlet can pass through the at least two conduits and exit via one or more openings bounded at least in part by a portion of one or more stand offs and a portion of the elongate body distal end. The exiting gas flows can provide at least one visual field improvement action for the visualization component. The sheath can further include one or more features configured to apportion gas between the at least two conduits. The sheath can further include one or more features distal to a sheath inlet to adjust the flow characteristics of the fluid discharged from the at least one conduit relative to the visualization component. The one or more features can adjust the relative velocity of the flow through the at least two conduits. The at least one conduit can include a first conduit and a second conduit. The first conduit can be configured to have a first flow of air and the second conduit can be configured to have a second flow of air, the first flow having a higher velocity than the second flow. The scope can further include a channel disposed completely within the sheath and in communication with an inlet at the sheath proximal end and having an outlet adjacent to the elongate body distal end. The outlet can be positioned adjacent to the exiting gas flows whereby the fluid provided via the outlet cooperates with the exiting gas flows to provide at least one visual field improvement action for the visualization component. The visual field improvement action can be one or more of: a gas flow pattern relative to the visualization component to remove condensation therefrom, a gas flow pattern relative to the visualization component to form an air barrier to reduce or minimize particles in the visual field of the visualization component and a gas flow pattern relative to the visualization component to facilitate removal of a fluid applied to the visualization component. The scope can further include a visualization component cable connected to the visualization component. The at least one attachment mechanism can be configured to releasably couple with the sheath using one or more snap fit features. The at least one attachment mechanism can be configured to releasably couple with the sheath using a gas tight friction fit. The at least one attachment feature can be configured to releasably couple with the sheath and an o-ring in a compression fit. The elongate body can be rigid, semi-rigid or flexible. The scope having an elongate body that can be flexible or semi-rigid can further include a handle having a steering mechanism for controlling a bend angle in a portion of the flexible or semi-flexible elongate body. The non-round cross section shape can have a substantially circular perimeter with at least a portion of the perimeter having at least one flattened portion. The non-round cross section shape can have a substantially circular perimeter with at least a portion of the perimeter having at least one non-circular portion. The non-round cross section shape can have a substantially ovoid perimeter with at least a portion of the perimeter having at least one flattened portion. The non-round cross section shape can have a substantially ovoid perimeter with at least a portion of the perimeter having at least one non-ovoid portion. The non-round cross section shape can have a substantially elliptical perimeter with at least a portion of the perimeter having at least one flattened portion. The non-round cross section shape can have a substantially elliptical perimeter with at least a portion of the perimeter having at least one non-elliptical portion. The non-round cross section shape can have a substantially triangular perimeter. The non-round cross section shape can have a substantially triangular perimeter with at least a portion of each corner of the triangular perimeter having at least one flattened portion. The non-round cross section shape can have a substantially triangular perimeter and each of the corners are rounded. The non-round cross section shape can have a substantially triangular perimeter and each of the corners are rounded and at least two of the corners have about the same radius of curvature. The non-round cross section shape can have a substantially circular perimeter with at least one cut out portion. The non-round cross section shape can have a substantially circular perimeter with a plurality of cut outs along the perimeter. The sheath can have an exterior wall having a substantially circular cross section shape and an interior wall forming a lumen sized, shaped, adapted and can be configured for a complimentary fit with the elongate body non-round cross section shape.

In general, in one embodiment, a sheath for use with a non-round scope includes a tube having a proximal end and a distal end and a gas inlet in the proximal end of the sheath. An interior wall of the tube defines an interior lumen extending from the proximal end to the distal end sized to receive the scope. The shape of the interior lumen is selected for a complementary fit with the exterior shape of the non-round scope. A first portion of the interior wall has a first shape, and a second portion of the interior wall has a second shape. When the scope is positioned within the interior lumen, the interior wall of the tube and the exterior wall of the scope are positioned such that a first channel is formed by the first portion of the interior wall and a first portion of the exterior wall of the scope and a second channel is formed by the second portion of the interior wall and a second portion of the exterior wall of the scope such that a gas introduced in a proximal end of the first and second channels flows across a distal face of the non-round scope.

This and other embodiments can include one or more of the following features. The first gas conduit can be in communication with a first gas outlet at the distal end of the sheath, and the second gas conduit can be in communication with a second gas outlet at the distal end of the sheath. The sheath can further include a visualization component in the scope distal end and an opening in a distal portion of the sheath sized for use with the visualization component. The sheath can have one or more stand offs such that when the scope is positioned within the sheath, the opening can be appropriately positioned relative to the visualization component and the one or more stand offs engage a portion of the scope distal face such that the gas flows from the first gas outlet and the second gas outlet can be directed towards the opening to further at least one visual field improvement action. The first channel can be configured to have a first flow of air, and the second channel can be configured to have a second flow of air. The first flow can have a higher velocity than the second flow. The sheath can further include a manifold in communication the gas inlet and with the first channel and the second channel. The sheath can further include one or more features distal to the gas inlet. The flow into the sheath from the inlet can be apportioned between the at least two conduits. The sheath can further include one or more features distal to the gas inlet to adjust the flow characteristics of the fluid discharged from the first channel and the second channel relative to the visualization component. The sheath can further include one or more features distal to the gas inlet to apportion the flow between the first conduit and the second conduit to adjust the flow characteristics of the gas flow relative to the visualization component. The one or more features can adjust the relative velocity of the flow through the first channel and the second channel. The exiting gas flows from the first channel and the second channel can provide at least one visual field improvement action for the visualization component. The visual field improvement action can be one or more of: a gas flow pattern relative to the visualization component to remove condensation therefrom, a gas flow pattern relative to the visualization component to form an air barrier to reduce or minimize particles in the visual field of the visualization component and a gas flow pattern relative to the visualization component to facilitate removal of a fluid applied to the visualization component. The sheath can further include one or more liquid stand offs positioned within the distal portion of the sheath. When the sheath is coupled to the scope, the one or more liquid stand offs can be adapted and configured to engage a portion of the distal portion of the scope such that a liquid flow from the liquid outlet can be directed towards the opening to further at least one visual field improvement action. The sheath can further include a liquid conduit within the sheath or formed as a third conduit between the sheath and the scope having a liquid outlet in relation to the scope distal end and an inlet at the sheath proximal end. The sheath can further include a channel disposed completely within the sheath and can be in communication with an inlet at the sheath proximal end and having an outlet adjacent to the scope distal end. The outlet can be positioned adjacent to the exiting gas flows such that the fluid provided via the outlet cooperates with the exiting gas flows to provide at least one visual field improvement action for the visualization component. The visual field improvement action can be one or more of: a gas flow pattern relative to the visualization component to remove condensation therefrom, a gas flow pattern relative to the visualization component to form an air barrier to reduce or minimize particles in the visual field of the visualization component and a gas flow pattern relative to the visualization component to facilitate removal of a fluid applied to the visualization component. The sheath can be adapted and configured for cooperative operation with an scope having an elongate body that is rigid, semi-rigid or flexible. The sheath can further include a handle coupled to the scope having a steering mechanism or a bending mechanism for controlling a bend angle in a portion of the flexible or semi-flexible elongate body of the scope. The sheath can further include at least one attachment feature adapted and configured using one or more snap fit features, a gas tight friction fit or an o-ring in a compression fit to releasably couple the sheath with the non-round scope inserted into the sheath. The non-round scope can have an elongate body that is rigid, semi-rigid or flexible. The non-round scope can have an elongate body that is flexible or semi-rigid, and the scope can further include a handle for use with the sheath and non-round scope combination having a bending or steering mechanism for controlling a bend angle in a portion of the flexible or semi-rigid elongate body.

In general, in one embodiment, a method of using the scope of any of the above includes: (1) inserting the scope into a human or animal body during a procedure; (2) visualizing a portion of the body using a visualization component of the scope; and (3) operating a view optimizing assembly to perform at least one visual improvement action.

This and other embodiments can include one or more of the following features. The visual field improvement action can be one or more of: a gas flow pattern relative to the visualization component to remove condensation therefrom, a gas flow pattern relative to the visualization component to form an gas barrier to reduce or minimize particles in the visual field of the visualization component and a gas flow pattern relative to the visualization component to facilitate removal of a fluid applied to the visualization component. The visual improvement action can be performed without removing the scope from the human or animal body during the procedure. The method can further include steering the scope by bending or orienting a flexible section of the scope. The visual improvement action can continue during the steering step. The method can further include supplying gas to the view optimizing assembly from a gas supply. The gas supply can be an insufflator. A portion of the human or animal body can be insufflated during the procedure.

Any of the above embodiments can include one or more of the following features. The sheath or scope or tip can be adapted and configured for use with a visualization component positioned within an scope distal end that can be one of 90 degrees, 45 degrees and 30 degrees. The visualization component can include a lens system. The visualization component can further include a solid state sensor, wherein the solid-state sensor can be selected from the following group: a Charge Coupled Device (CCD); an Intensified Charge Coupled Device (ICCD); an Electron Multiplying Charge Coupled Device (EMCCD); and a Complementary Metal Oxide Semiconductor (CMOS) device. The visualization component can be a part of a tip face of a sterilizable elongate body of a non-round scope. The visualization component of a non-round scope can include a lens system having a plurality of lens that together form an image with a field of view of between 60 and 140 degrees. The non-round scope, the sheath and the visualization component can be adapted and configured for carrying out a procedure selected from the following group: (a) a gastroscopy procedure by forming an image with a field of view of 120 to 140 degrees; (b) an ERCP procedure by forming an image with a field of view of the camera head of the invention 120 to 140 degrees in a motherscope and by forming an image with a field of view of 100 degrees in a baby scope; (c) a colonoscopy procedure by forming an image with a field of view of 120 to 140 degrees; (d) a gynecology procedure by forming an image with a field of view of 100 to 120 degrees; (e) a bronchoscopy procedure by forming an image with a field of view of 80 to 100 degrees; (f) an ENT procedure by forming an image with a field of view of 80 to 100 degrees; and (g) a transgastric procedure by forming an image with a field of view of 120 to 140 degrees in the motherscope and by forming an image with a field of view of 100 to 120 degrees in the baby scope. The visualization component can include a sensor having a diagonal size in the range from approximately 0.5 mm, 1 mm, 1.5 mm, 2 mm, 2.5 mm, 3 mm, or 3.5 mm, or 4 mm. The first and second channels can be together configured to direct air over the lens in a vortex.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a perspective view of an endoscopic or laparoscopic system including a tip cap having a view optimizing assembly.

FIG. 2A is a close up perspective view of the scope of FIG. 1.

FIGS. 2B and 2C are left and right side cross section views, respectively, of FIG. 2A without the tip cap.

FIG. 3 is a cross section view of a handle having a gas dividing manifold.

FIG. 4A illustrates a perspective view with the tip cap removed from the scope of FIG. 2A.

FIG. 4B is a top down view of the tip engagement region in FIG. 4A.

FIG. 5A is an enlarged perspective view of the distal end of the laparoscope in FIG. 2A with the tip cap coupled to the tip engagement region.

FIG. 5B is a section view of the distal end of the laparoscope in FIG. 2A.

FIGS. 6A and 6B are left and right top perspective views, respectively of the tip cap of FIGS. 4A and 5A.

FIGS. 6C, 6D and 6E illustrate various section views of the tip cap illustrated in FIGS. 6A and 6B.

FIGS. 7A and 7B illustrate a perspective end and side views of the distal portion and tip engagement region of an endoscope having a visualization component, three gas line outlets and one fluid outlet in a tip face.

FIG. 7C is an end perspective view of a tip cap adapted for used with the endoscope of FIGS. 7A and 7B coupled to the tip engagement region of the scope.

FIG. 8A illustrates a perspective end view of the distal portion and tip engagement region of an endoscope having a visualization component, one gas line outlet and one fluid outlet in a tip face. The visualization components includes a camera lens and integrated LED lighting array as best seen in the view of FIG. 8B where the distal face has been removed.

FIG. 9 is a perspective view of another embodiment of a scope having a tip cap thereon.

FIG. 10 is a perspective view of an endoscope system including a semi-rigid or flexible elongate body endoscope.

FIG. 11A illustrates an end view of one embodiment of a tip cap.

FIG. 11B illustrates an end view of another embodiment of a tip cap.

FIG. 11C illustrates an end on view of another embodiment of a tip cap.

FIG. 12 illustrates a cross sectional view of a tip cap removed from the tip engagement region of an endoscope.

FIG. 13A illustrates a cross sectional view of a tip engagement region of an endoscope including a portion of an imaging component.

FIG. 13B illustrates a tip cap adapted to releasably couple to the tip engagement region of FIG. 13A with the tip engagement region in position prior to engagement.

FIG. 14A is a perspective view of a rigid non-round endoscope having a round sheath therearound.

FIG. 14B is a close up perspective view of the non-round scope and sheath combination shown in FIG. 14A showing the details of a handle, a gas inlet, a fluid inlet and video or optics cable connection port.

FIGS. 14C and 14D are distal and bottom up views, respectively of the distal end of the endoscope and sheath combination of FIG. 1A.

FIG. 15A is the non-round endoscope of FIG. 14A without the sheath.

FIG. 15B is an enlarged isometric view of the distal end of the non-round endoscope of FIG. 15A.

FIG. 15C is an enlarged view of the proximal end of the non-round scope in FIG. 15A.

FIG. 15D is an enlarged view of the various features in the proximal end of the scope.

FIG. 16A is an isometric view of the distal end of the non-round endoscope and sheath combination of FIG. 14A.

FIG. 16B is cross section view of the isometric view of FIG. 16A taken proximal to the distal end showing the complementary fit of the non-round scope exterior surface and the interior lumen of the sheath.

FIG. 16C is cross sectional view of the view of FIG. 16B with the non-round scope removed. Indicated in this view are portions of the sheath wall shaped to engage with the scope external wall and portions of the sheath wall shaped to form one or more channels or conduits.

FIG. 16D is a bottom up view of the distal end of an endoscope having a plate between the sheath and scope face.

FIG. 17A is an isometric view of the distal end of a scope and sheath combination in illustrating the section lines for the views shown in FIGS. 17B and 17C.

FIG. 17B is a section view of the distal end of the non-round scope and sheath combination taken along the longitudinal axis of the scope/sheath combination as indicated in FIG. 17A.

FIG. 17C is a section view of the distal end of the non-round scope and sheath combination taken along the transverse or short axis of the scope sheath combination as indicated in FIG. 17A.

FIG. 18 is an isometric view of the distal end of the scope-sheath combination of FIG. 17A illustrating exemplary gas flow paths created when the scope-sheath combination is engaged and gas is supplied to the sheath gas inlet.

FIG. 19A is an exemplary non-round scope cross section having a partially flattened ovoid or elliptical shape.

FIG. 19B is an exemplary non-round scope cross section having a D-shape.

FIG. 19C is an exemplary non-round scope cross section having a generally triangular shape similar to that of FIG. 15B with at least one corner flattened.

FIG. 20A is an exemplary non-round scope cross section having two cut outs that form conduits when the scope is inserted into a complementary sheath.

FIG. 20B is an exemplary non-round scope cross section having three cut outs that form conduits when the scope is inserted into a complementary sheath.

FIG. 20C is an exemplary non-round scope cross section having three cut outs that form conduits when the scope is inserted into a complementary sheath.

FIG. 21 is a section view of a non-round scope and sheath combination showing the formation of channels along the scope bounded by an interior wall of the sheath and a shaped portion of the exterior wall of the scope.

FIG. 22 is a section view of another exemplary non-round scope and sheath combination.

FIG. 23A illustrates an end view of one embodiment of a deflector assembly.

FIG. 23B illustrates an end view of another embodiment of a deflector assembly.

FIG. 23C illustrates an end on view of another embodiment of a deflector assembly.

FIG. 24A is an isometric view of the distal end of a non-round endoscope and sheath combination of FIG. 14A without a separate fluid conduit in the sheath.

FIG. 24B is cross section view of the isometric view of FIG. 24A taken proximal to the distal end.

FIG. 24C is cross section view of the view of FIG. 24B with the non-round scope removed.

FIG. 25A is a cross-section of a scope and deflector assembly showing formation of a vortex of gas over the lens of the scope.

FIG. 25B is a cross-section of a scope and deflector assembly showing formation of a vortex of gas over the lens of a scope.

FIG. 25C shows the formation of two different gas flows over a scope lens.

FIG. 26A shows an exemplary scope with a deflector assembly.

FIG. 26B shows an exemplary configuration of stand-offs relative to a gas lumen to provide gas flows of different velocities.

FIG. 26C shows an exemplary configuration of stand-offs relative to two gas lumens to provide gas flows of different velocities.

FIG. 26D shows an exemplary configuration of stand-offs relative to a single gas supply from a plenum to provide gas flows of different velocities.

### DETAILED DESCRIPTION

Described herein are various view optimizing assemblies for use with a scope, such as an endoscope, laparoscope, or other surgical scope. The view optimizing assembly can be configured to extend distally past the lens of the scope and direct air thereacross, thereby improving visualization through the lens. The view optimizing assembly thus facilitates inter-operative defogging, surgical debris reflection, and cleaning of the scope lens during minimally invasive surgery, while also maintaining visualization of the surgical site.

As described herein, the view optimizing assembly can be: (1) a sheath that extends over the scope; (2) a tip cap that attaches to the end of the scope; (3) and/or can be integrated with the scope. The term “scope” as used herein can be interchangeable with “laparoscope” or “endoscope.”

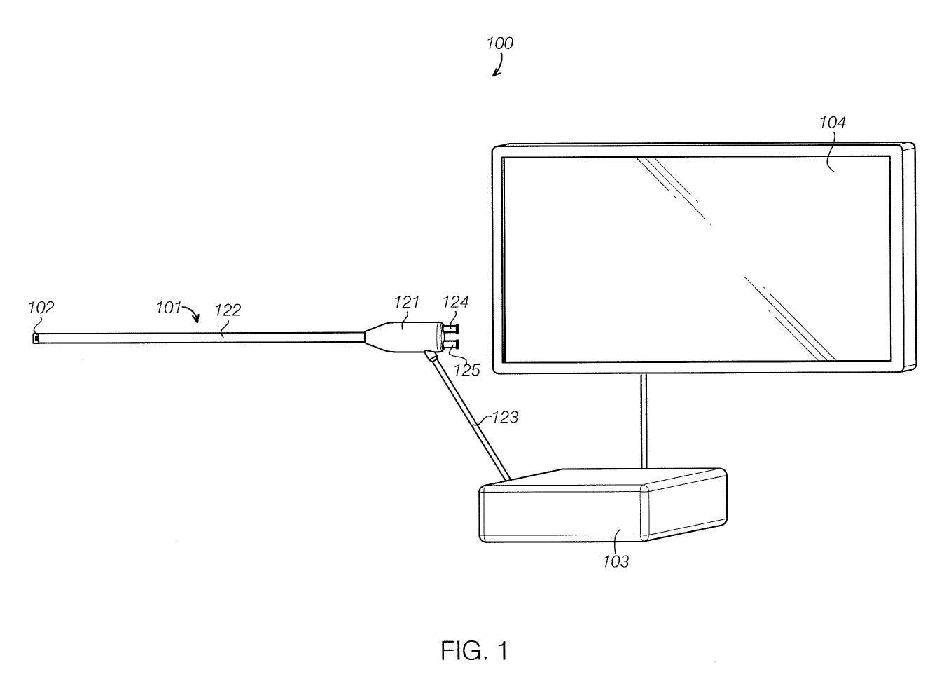

FIG. 1 is a perspective view of an endoscopic or laparoscopic system 100 including a re-useable scope 101 with a handle 121, an elongate shaft 122, and a view optimizing tip cap 102. A gas supply port 124 and a fluid supply port 125 can be configured to attach to a source of gas and a source of fluid, respectively, for use during laparoscopic or endoscopic procedures. The scope 101 is attached to a camera processor 103 through a video or optics cable 123, which is in turn attached to a high definition monitor 104 for displaying images obtained by the scope 101.

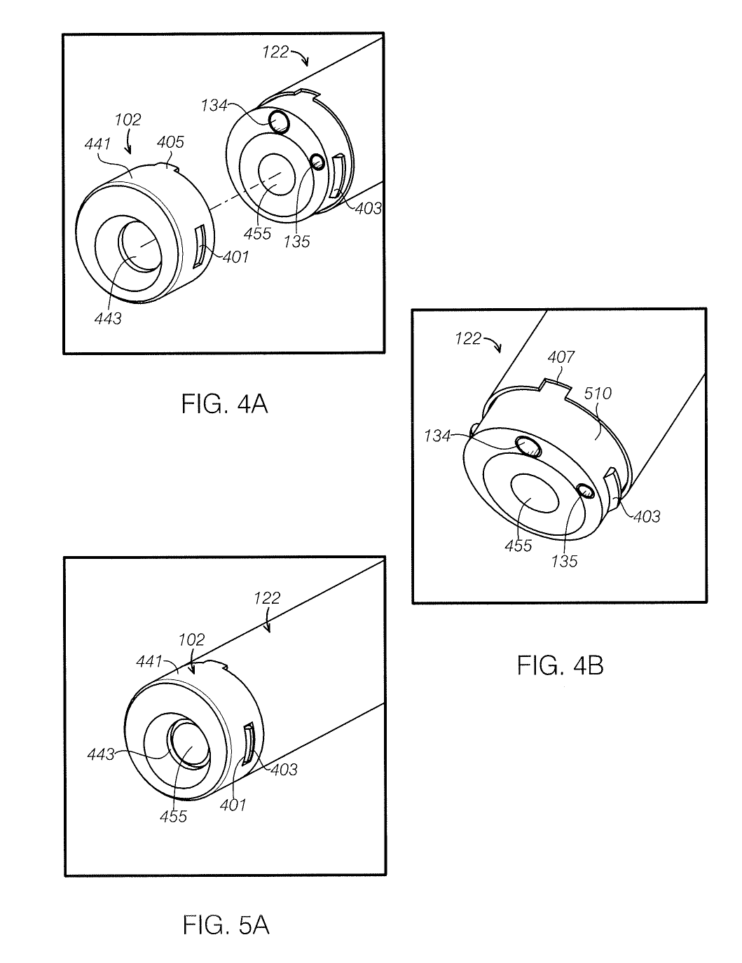

As can best be seen in FIG. 4A, the tip cap 102 can include an annular body 441 with an opening 443 extending therethrough. The tip cap 102 can further include an attachment mechanism 401 and/or a mating mechanism 405 for attachment and/or mating with the shaft 122. As is further described below, the tip cap 102 can also include a plurality of dividers, stand-offs, and or lumens on the inner surface thereof configured to direct gas and/or fluid over the lens of the scope. In use, the tip cap 102 improves visualization through the scope by directing the air in a desired flow, such as in a vortex, over the lens.

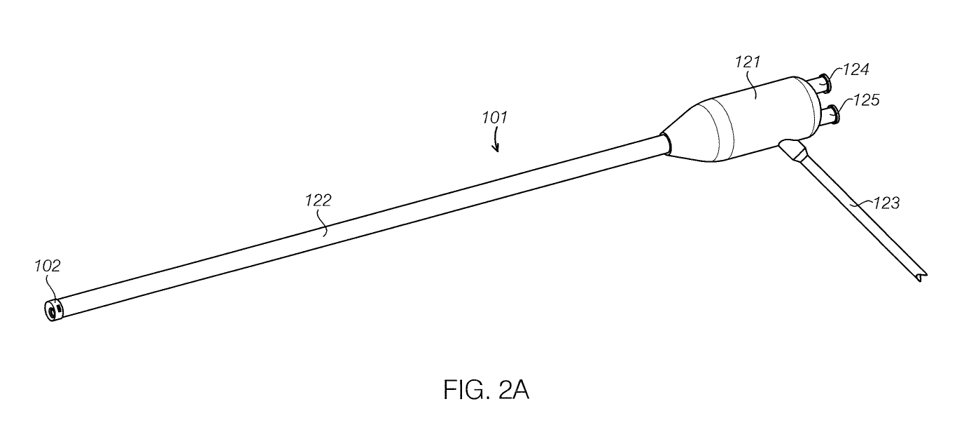

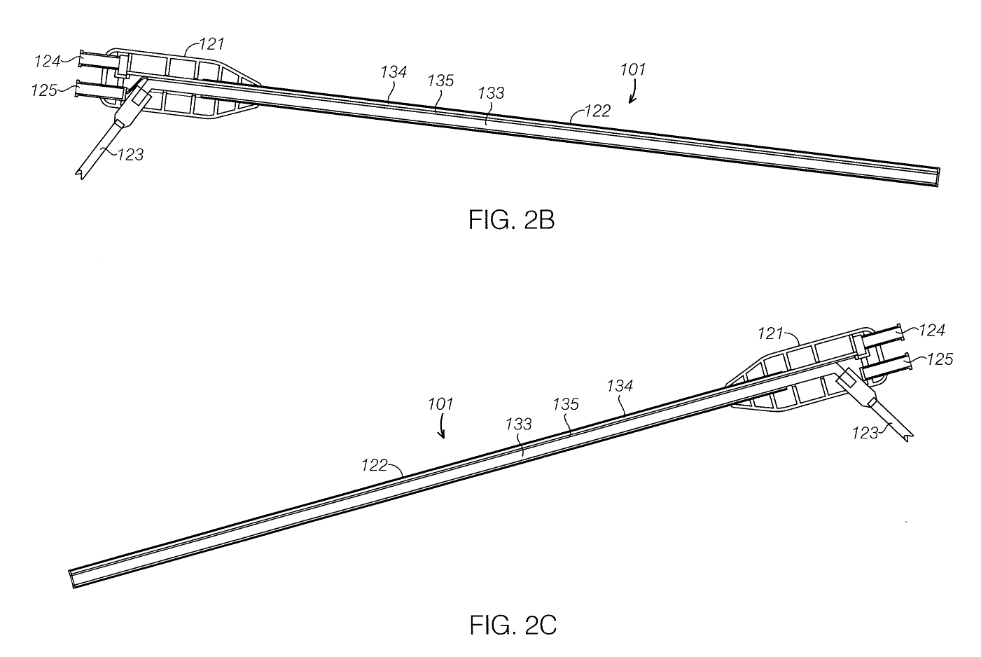

FIGS. 2A-2C show the scope 101 in more detail. As shown, the scope 101 can include a gas conduit 134 connected to the gas supply port 124 and extending the length of the shaft 122, a fluid conduit 135 connected to the fluid port 125 and extending down the length of the shaft 122, and an optics conduit 133 connected to the optics cable 123 and extending down the length of the shaft 122. As will be described further below, the gas and fluid conduits can be configured to connect to the tip cap 102 for directing air across the lens of the scope 101.

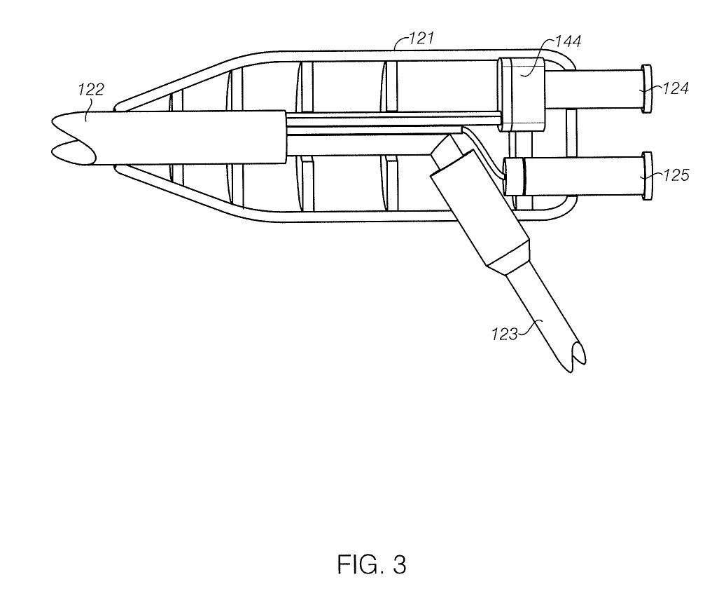

Referring to FIG. 3, in some embodiments, the handle 121 can include a gas dividing manifold 144 attached to the gas supply port 144. The gas dividing manifold 144 can be configured to divide the gas into a plurality of gas streams (for example, to send down a plurality of conduits within the shaft 122). The velocity of air through each of the conduits can be adjusted so as to achieve the desired airflow over the lens, such as to form a vortex, as will be described further below. In some embodiments, the gas dividing manifold 144 is configured similar to the manifold described in U.S. Patent Application Publication No. 2012/0197084 to Drach et al., titled “SYSTEMS AND METHODS FOR OPTIMIZING AND MAINTAINING VISUALIZATION OF A SURGICAL FIELD DURING THE USE OF SURGICAL SCOPES,” filed Aug. 4, 2011 (the “'084 application”), the entirety of which is incorporated herein by reference.

Referring to FIGS. 4A-4B, the tip cap 102 can be configured to be attached and detached from the shaft 122. Thus, the tip cap 102 can include a snap feature or attachment mechanism 401 while the shaft 122 can include a mating attachment mechanism 403 on a tip engagement region 510 (i.e., the region of the shaft configured to interact with or mate with the tip 102). The attachment mechanisms 401/403 can be any suitable elastic integral mechanical attachment or interlock sufficient to maintain the coupling and consistent with the use of the endoscope, such as for example: cantilever hooks, cantilever holes, window snaps, annular snaps, leaf-spring snaps, ball and socket, post and dome, compression hooks, compression traps, compression beams, bayonet finger snaps, torsion snaps, integral spring tabs, spring plugs, spring clips, snap slides, and quick release fasteners. In some embodiments, the tip cap 102 can include a mating feature 405 configured to engage with a mating feature 407 on the shaft 122, for example to ensure proper alignment. The mating features 407/122 can be, for example, a tab, slot, notch, or indent/detent. The diameter of the scope engagement portion 510 can be less than the diameter of the rest of the shaft 122 to allow for engagement with the tip cap 102 while still providing a smooth outer diameter scope. In some embodiments, the diameter of the scope engagement portion 522 is at least 0.030 inches less than a diameter of the rest of the shaft 122.

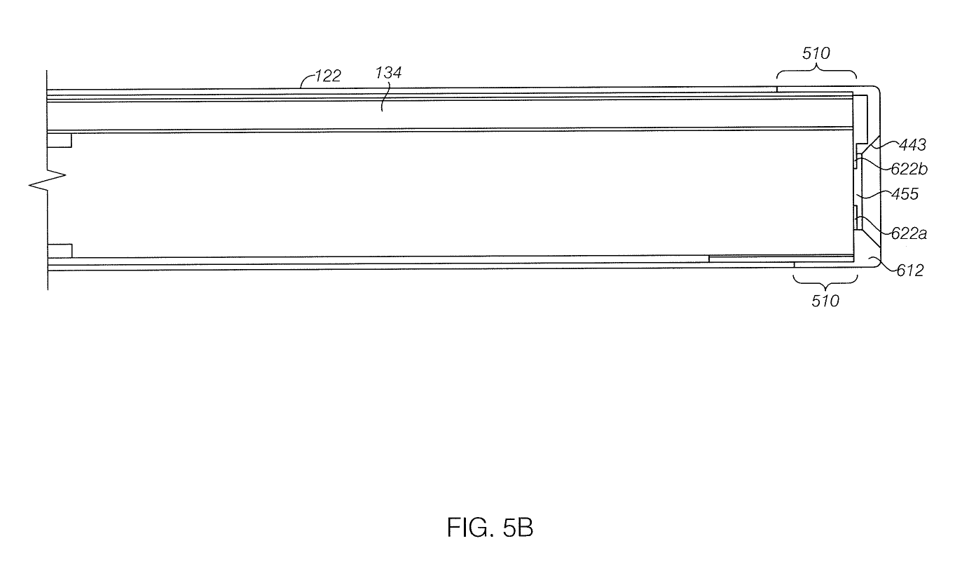

Referring to FIGS. 4A-4B and 5A-5B, when the tip cap 102 is attached to the shaft 122, it can be configured such that the opening 443 is positioned around the lens 455 of the scope, thereby leaving the lens clear for imaging. Further, the gas conduit 134 and fluid conduits 135 of the scope 101 can be positioned relative to features (such as stand-off 612) on the inside of the cap 102 so as to direct air across the lens 455, i.e., through nozzles or gas outlets 618a,b. The outlets 622a,b can extend approximately 0.005 inches to 0.010 inches off of the distal end of the scope 101.

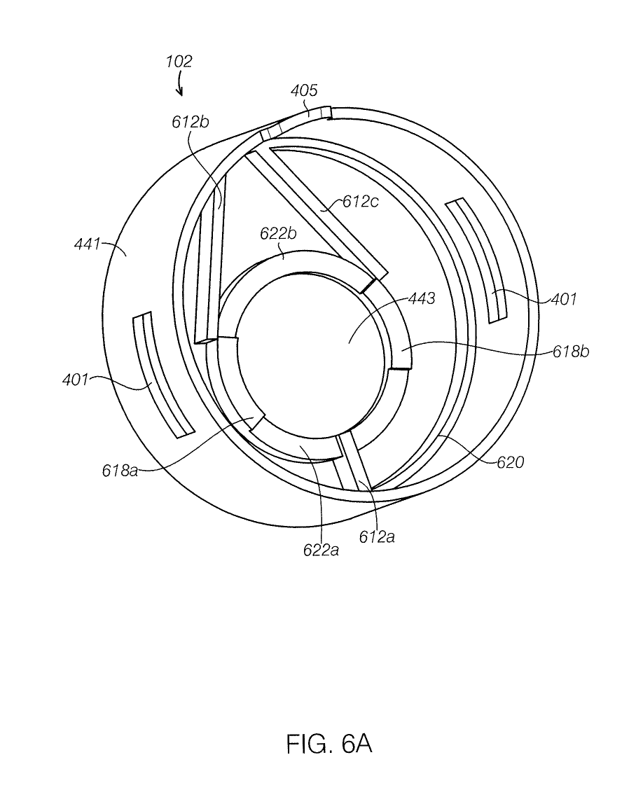

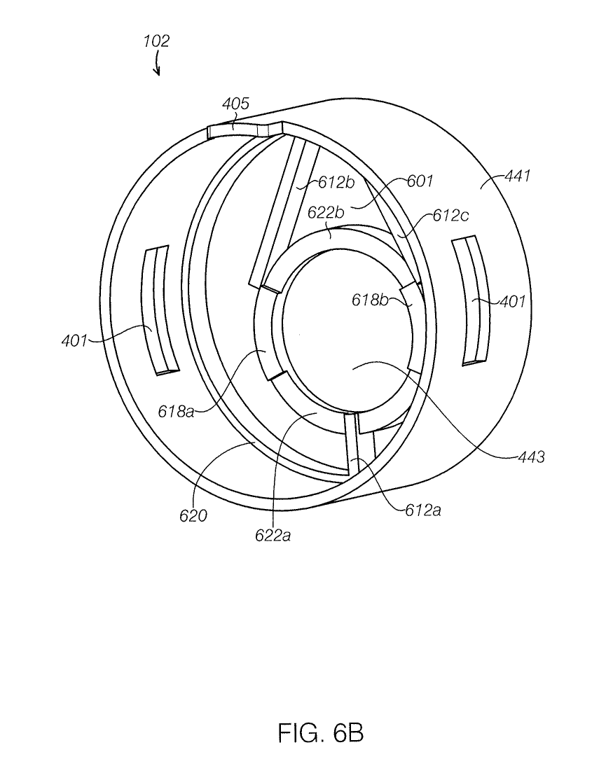

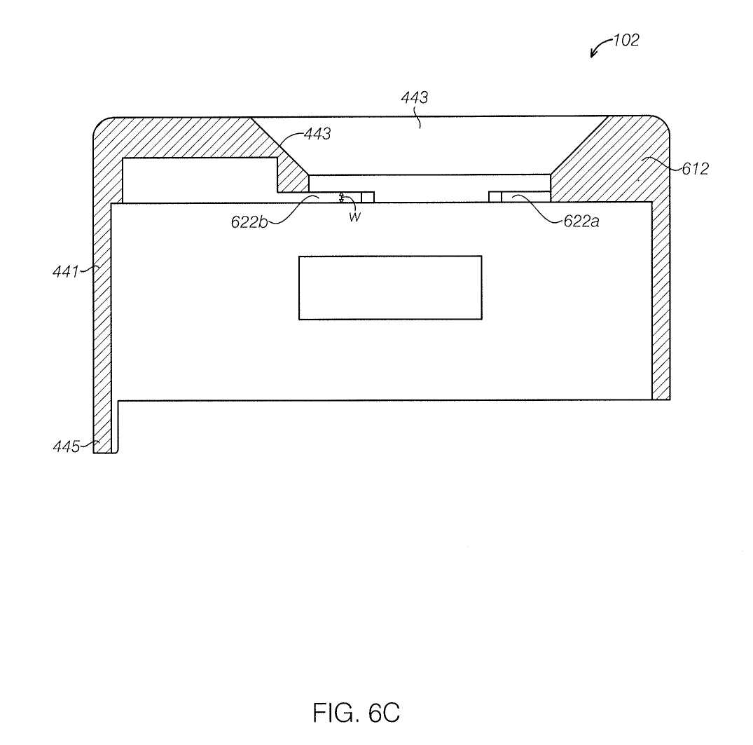

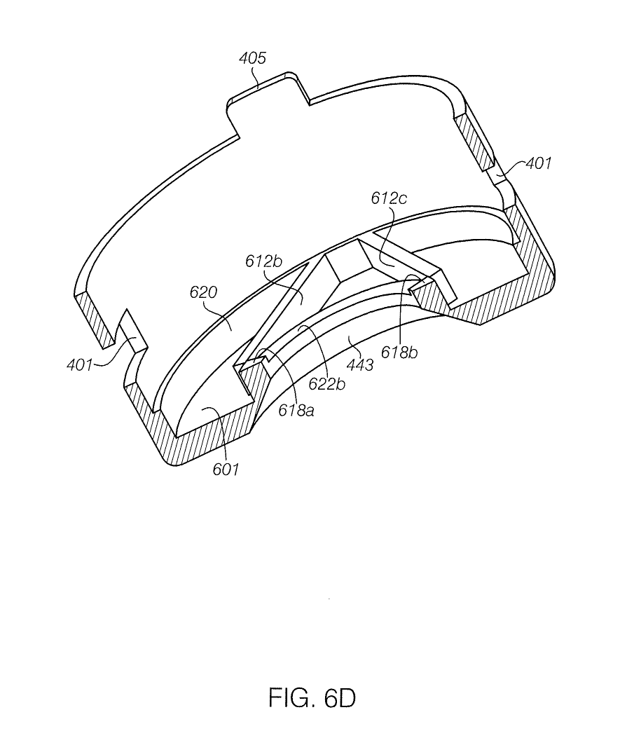

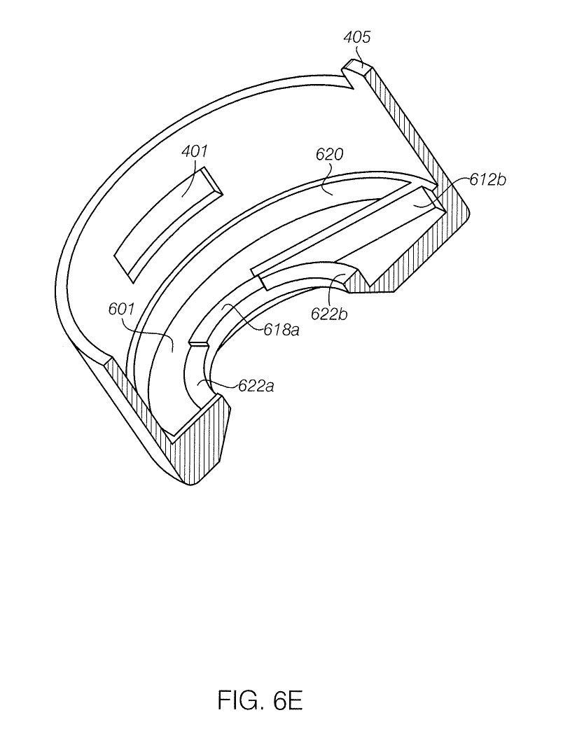

The inner distal face of the tip cap can thus function as a deflector assembly. The deflector assembly projects beyond the distal end of the scope and also a predetermined distance towards the central axis of the scope and the lens. The deflector assembly overhangs the distal face of the scope by a prescribed transverse distance sufficient to change the direction of gas flowing axially through conduits into a non-axially, transverse path across the laparoscopic lens. The distance of the deflection width does not extend to the point that it obstructs the field of the view of the laparoscopic lens. The deflector assembly also projects axially beyond the distal terminus of the scope by a prescribed axial distance, defining an air channel 622 or nozzle (see FIG. 6C). The nozzle can have a width w of between 0.005 inches and 0.010 inches. The deflector assembly is sized and configured to direct the portion of the air/gas that is conveyed through the conduits in a prescribed flow path and flow velocity across the lens, as will be described in greater detail later.

FIGS. 6A-6E show exemplary features on the inside of the tip cap 102 configured to direct air across the lens. The distal inner face includes a plurality of stand-offs 612a-c, 618a-b, and 620 that act as gas diverters to flow gas towards the opening 443 (and thus over the lens when the cap is engaged with the scope). In this embodiment, the stand-off 620 extends around the outer perimeter of the face 601. Stand-offs 618a-b extend around the perimeter of the opening 443 while allowing channels 622a,b therethrough for air to flow into the opening 443. Further, stand-offs 612a-b extend radially from the opening 443 to the perimeter stand-off 620.

The stand-offs or gas diverters redirect gas or fluids introduced into the tip cap 102 towards the opening 443. In one embodiments, the stand-offs direct the air in such a way as to form a vortex over or proximate to the lens, as described further below.

The fit between the tip cap 102 and the tip face/tip engagement region 510 is adapted and configured to prevent gas loss and seal relative thereto either through the engagement alone or with an additional seal mechanism. As a result, most of the gas or fluid introduced into the tip cap 102 is directed through the gas channels 622a,b to opening 443 and over the lens.

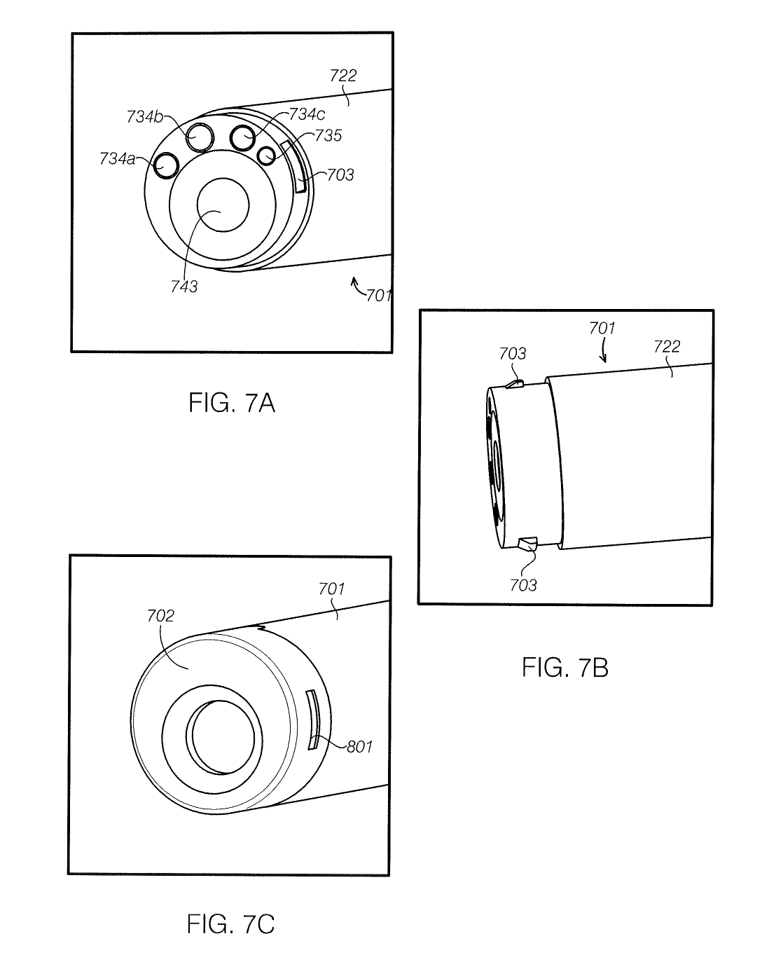

FIGS. 7A-7C illustrate another embodiment of a scope 701 having a tip cap 702 thereon for visual enhancement. The scope 701 and cap 702 are similarly configured to the scope 101 and tip cap 102 (with shaft 722, lens 743, attachment mechanisms 703/801). In contrast to the scope 101, however, distal region of the scope 701 includes three gas conduits 734a-c and one fluid outlet 735. In some embodiments, these different gas conduits 734a-c can be sized and configured to have gas flow of differing velocities flowing therethrough so as to form a vortex over or proximate to the lens. In other embodiments, the velocity gas flow through each of the conduits 734a-c can be the same for all three conduits, and the distal cap 702 can be used to separate the gas into varying velocities, as will be described further below.

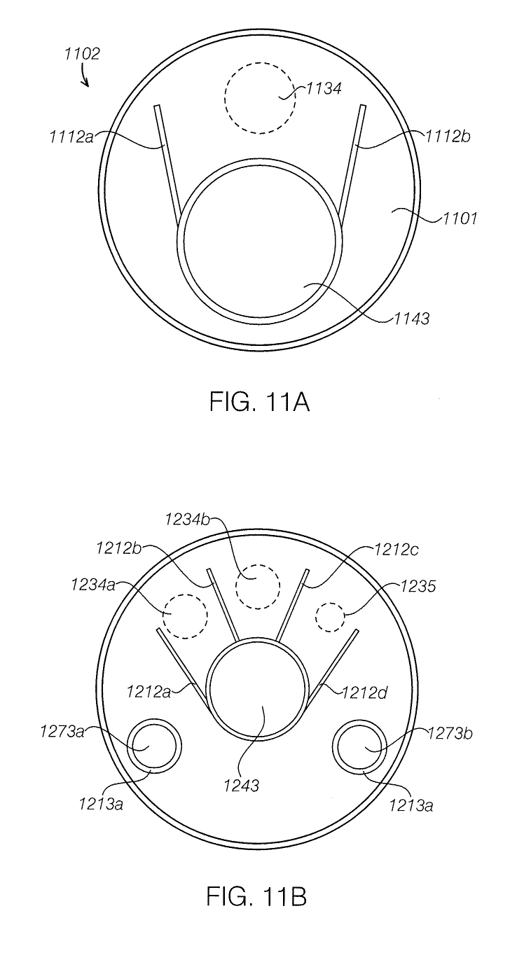

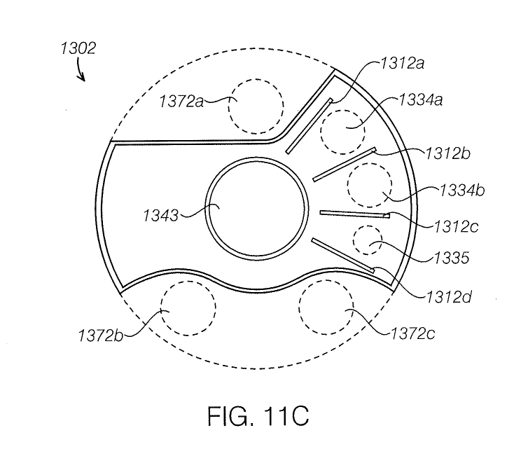

FIGS. 11A-11C show different embodiments of the interior of a tip cap with relative placement of gas or fluid conduits (from the elongate body) shown in dotted lines. FIG. 11A shows a tip cap 1102 configured so as to attach to a scope and cover substantially all of the tip face with the exception of the lens (via the opening 1143). Cap 1102 includes a single gas outlet 1134 at the tip face. Stand-offs 1112a and 1112b extend radially from the opening 1143 on either side of the gas outlet 1134 to direct gas towards the opening 1143.

FIG. 11B shows a tip cap 1202 configured so as to attach to a scope and cover substantially all of the tip face with the exception of the lens (via the opening 1243) and the working channel (via working channel openings 1273a,b). Cap 1202 includes a pair of gas outlets 1234a,b and a fluid outlet 1235. Stand-offs 1112a,b,c,d extend radially from the opening 1243 and separate each of the outlets 1234a,b and 1235 to direct gas and/or fluid towards the opening 1243. Additional stand-offs 1213a,b are provided around the working channel openings 1272a,b to seal the working channel (for allowing instruments to pass therethrough) and to maintain gas integrity within the tip cap.

FIG. 11C shows a tip cap 1302 configured so as to attach to a scope and cover only a portion of the tip face to provide the desired visual field improvement actions. In contrast to FIG. 11B, the three working channels 1372a,b,c are excluded from tip coverage. The tip cap 1302 thus has a non-round cross-section to exclude the working channels 1372a,b,c. The tip cap 1302 includes an opening 1343, a pair of gas outlets 1312a and 1312b, and a fluid outlet 1335. Stand-offs 1112a,b,c,d extending radially from the opening 1343 and separate each of the outlets 1334a,b and 1335 to direct gas and/or fluid toward the opening 1343.

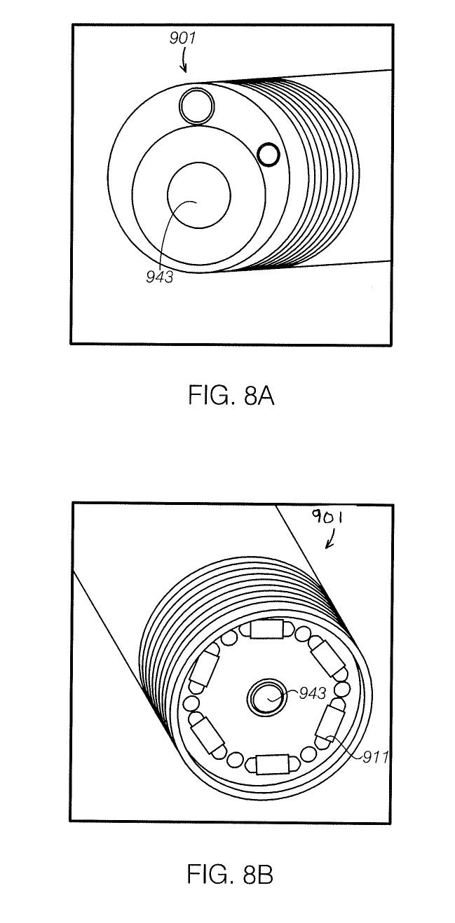

FIGS. 8A and 8B illustrate alternative tip engagement regions of a scope 901 for use with a view optimizing tip cap, as described herein. The engagement region of the scope 901 includes a screwing attachment mechanism 909 rather than a tab and slot mechanism. Further, FIG. 8B shows an LED light array 911 arranged about the lens 943.

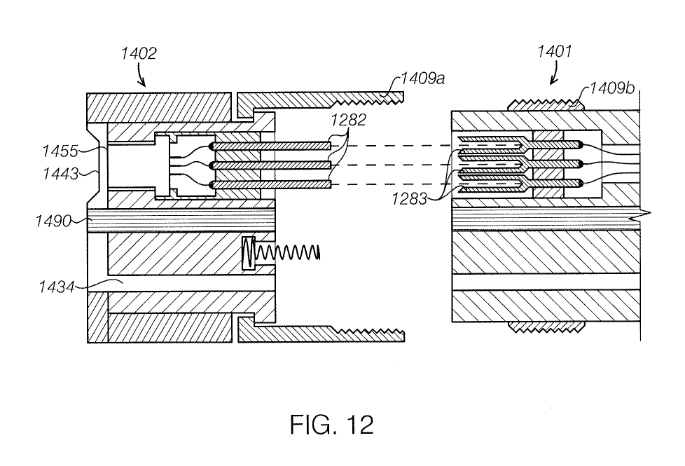

FIG. 12 is a cross section of an exemplary tip cap 1402 removed from the tip engagement region of a scope 1401. The tip cap 1402 is configured to screw onto the scope 1401 through a screw-type engagement mechanism 1409a,b. The tip cap 1402 further includes an opening 1443 configured to expose the lens 1455 therethrough and a connection to a lighting array 1490. A gas conduit 1434 extends through the tip cap 1402 so as to allow gas to flow over the lens 1443. As shown in FIG. 12, the tip cap 1402 can include electric connections 1282 configured to mate with electrical connections 1283 within the scope for operation of the visualization component. Additional details of the modified endoscope illustrated in FIG. 12 may be obtained by reference to Figure. 8 in U.S. Pat. No. 7,435,218 to Krattiger et al., titled “OPTICAL INSTRUMENT, IN PARTICULAR AN ENDOSCOPE, HAVING AN INTERCHANGEABLE HEAD,” FILED Oct. 27, 2003, incorporated herein by reference.

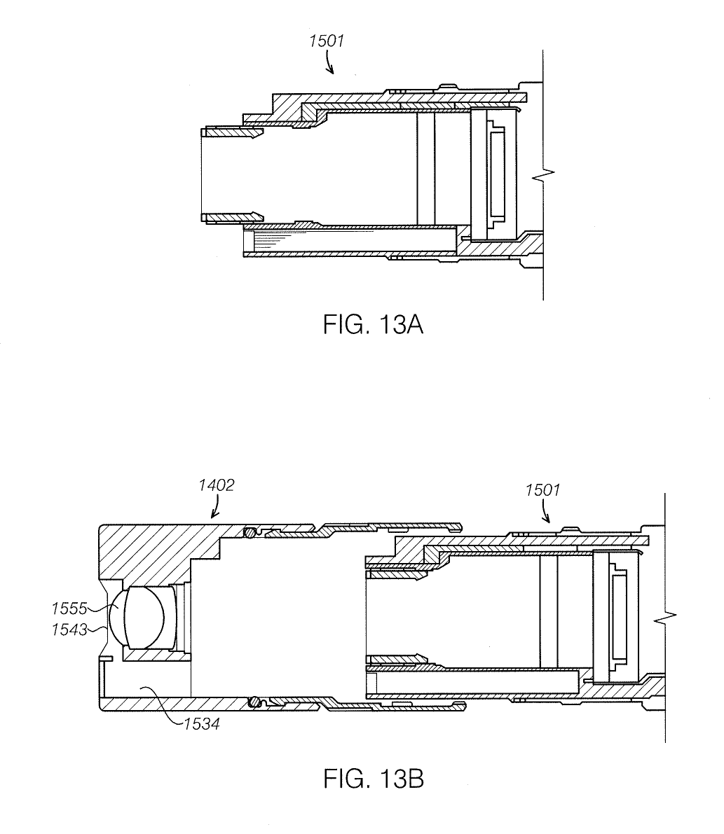

FIG. 13A illustrates a cross section view of a tip engagement region of an endoscope 1501 including a portion of an imaging component. FIG. 13B illustrates a tip cap 1502 adapted to releasably couple to the tip engagement region of FIG. 13A. In this embodiment, the tip cap 1502 includes a portion of the visualization system (i.e., lens 1555) and opening 1543. The tip cap 1502 further includes a gas channel 1534 for providing one or more of the visual field improvement actions. The tip engagement region of the scope likewise includes a gas supply conduit and optical connections to mate the imaging component and the lens.

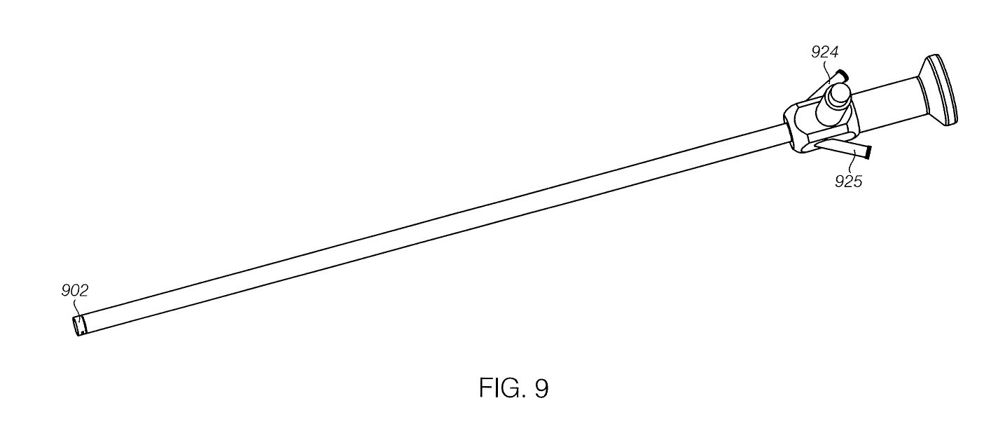

FIG. 9 is a perspective view of another embodiment of a scope having one or more gas and fluid conduits within the body (connected via ports 924, 925) extending down to a distal tip cap 902.

There are a number of advantages to providing visual field improvement to a re-usable scope, such as with the tip cap as described herein. For example, the tip caps may be disposable. Moreover, the tip caps may be designed to accommodate a variety of combinations of diameter and angles as well as working channels or other ports, depending upon the design characteristics of a particular endoscope. In each of these different configurations, the tip cap is adapted and configured direct the gas over the lens (i.e., one or more visualization components) from a single gas supply lumen or from multiple gas supply lumen in the endoscope.

Still further, re-usable scopes (for use with the tip cap) can be readily decontaminated and re-sterilized. Further, the scope 101 can be made (or interchanged with other scopes) of different lengths depending upon application or surgical need. Similarly, the distal end of the scope 101 can be made (or interchanged with other scopes) in different angles (0, 30, 45 degrees) and in different diameters (5 mm, 10 mm). In each case, the corresponding tip cap 101 can be adapted and configured for use with the angle and diameter.

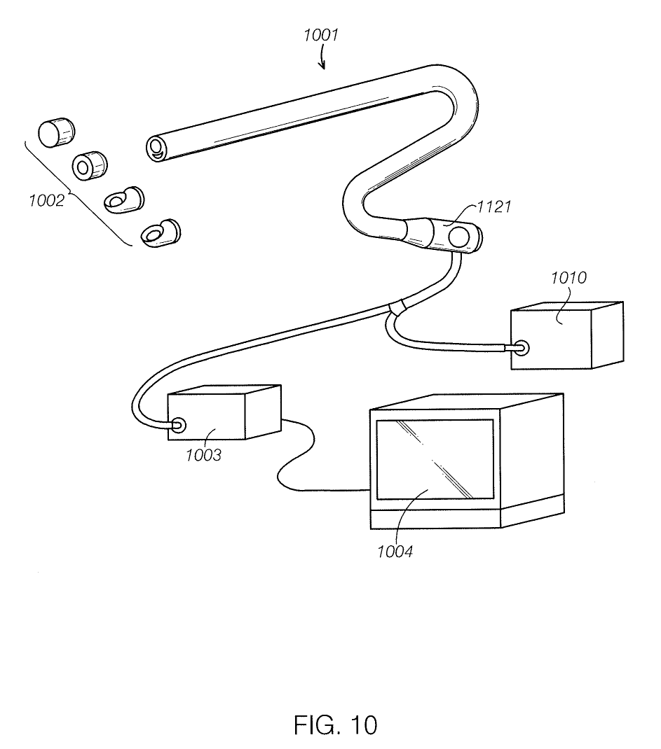

Advantageously, the tip cap can also be used with a scope that is rigid, flexible, or semi-rigid, as the stiff cap can be placed on portions of the scope that do not bed, thereby not interfering with any flexibility and/or steering. FIG. 10 is a perspective view of an endoscope system 1000 similar to FIG. 1 including a semi-rigid or flexible elongate scope 1001 having a handle 1121 with a steering or bending control for the semi-rigid or flexible body. Also shown is an imaging processor 10003 and associated display 1004. The system 1000 further includes a gas supply 1010 for use in providing the one or more visual field improvement actions. The endoscope system also has multiple different and interchangeable tip caps 1002, each of which may be releasably coupled to the tip engagement region of the semi-rigid or flexible elongate body endoscope. The interchangeable tips can function, for example, to direction air to different sides of the scope, to flow only fluid thereacross, and/or perform different visualization enhancement improvement actions from one another.

Additional details of the modified endoscope system of FIG. 10 and the modified endoscope embodiments of FIGS. 13A and 13B may be obtained by reference to FIGS. 20, 6 and 7, respectively, of U.S. Pat. No. 6,206,825 to Tsuyuki, titled “ILLUMINATION SYSTEM FOR ENDOSCOPES AND AN ENDOSCOPE HAVING THE ILLUMINATION SYSTEM,” filed Jan. 15, 1999, incorporated herein by reference.

Any of the tip caps described herein may be formed partially or completely from an x-ray detectable material. Alternatively, one or more radio-opaque or x-ray detectable markers may be positioned on, in or within a portion of the tip cap. The position, number, of or size of the markers can be selected in order to aid in location of the tip cap while in use with an endoscope during a medical procedure.

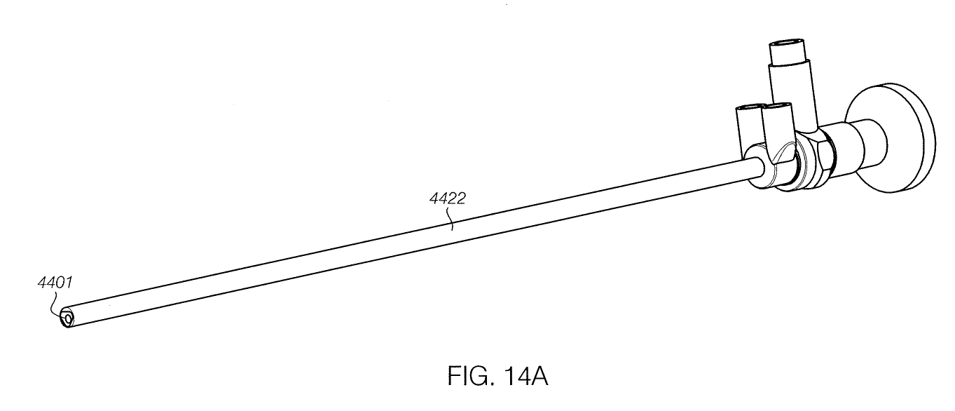

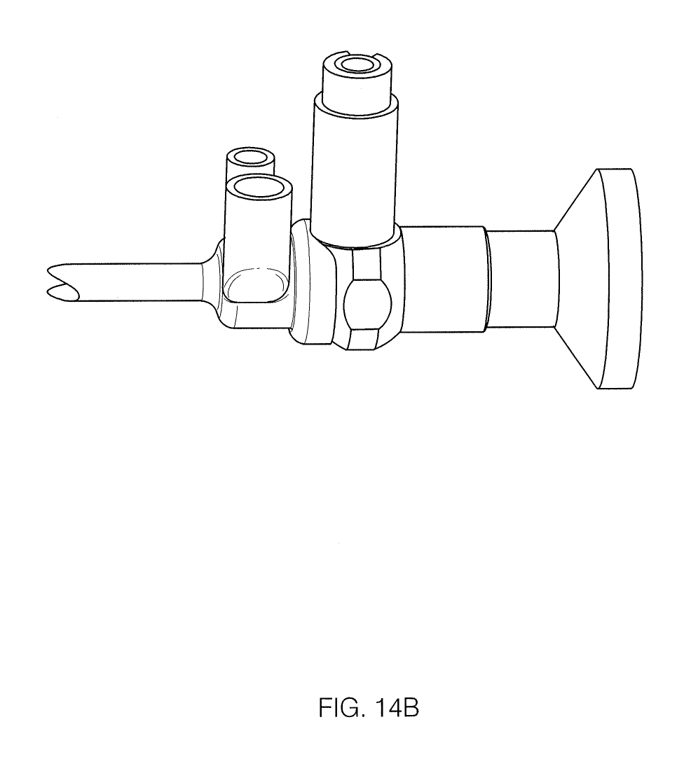

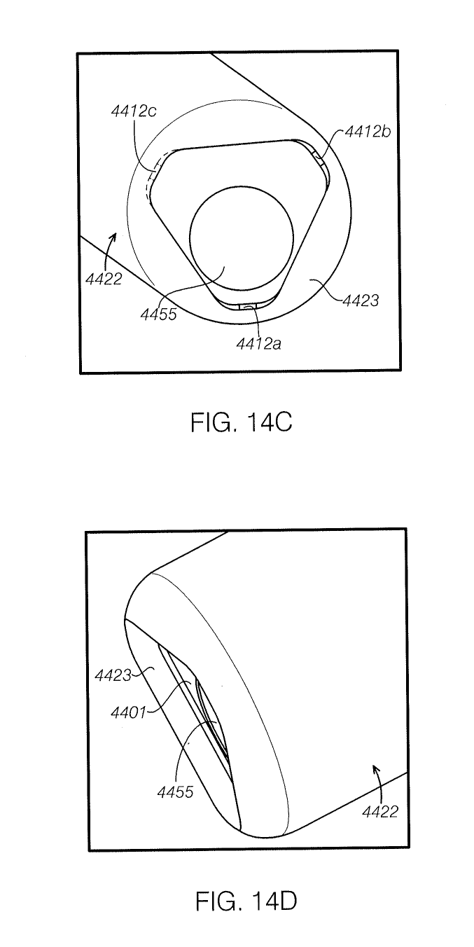

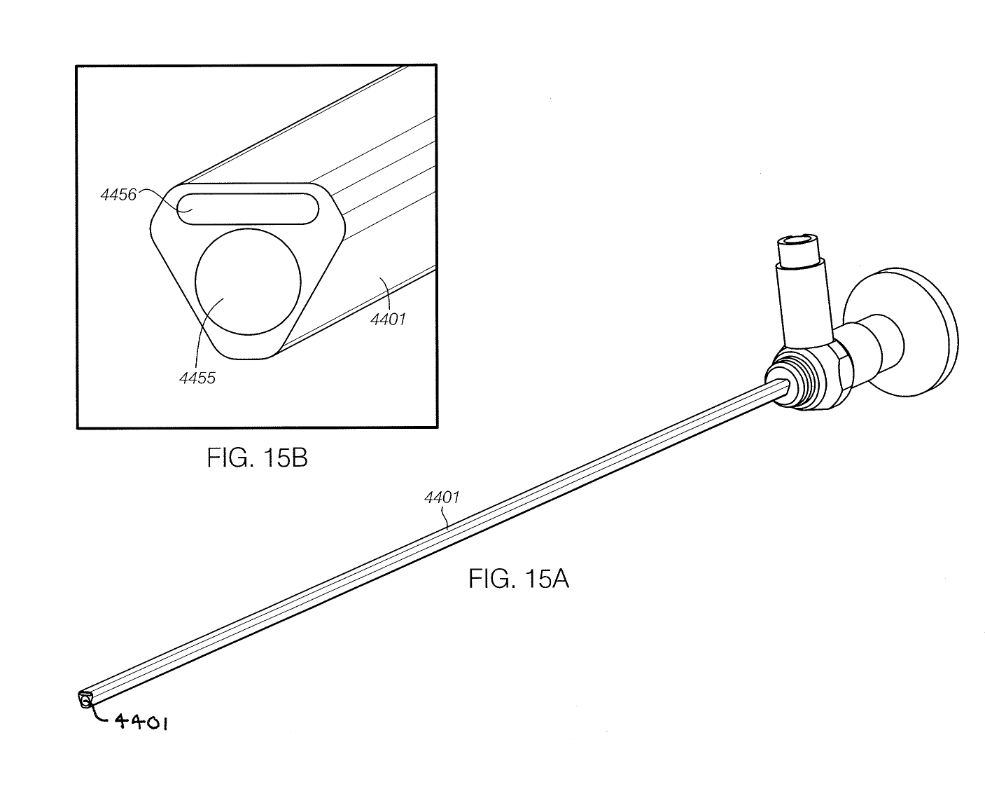

FIG. 14A is a perspective view of an endoscopic system having a re-useable non-round endoscope 4401 with a round complimentary sheath 4422 extending therearound. The non-round scope 1401 may be configured for use with a visualization system, camera processor and an associated high definition monitor, similar to as shown in FIG. 1. The non-round aspect of the scope means that the scope has an overall exterior cross sectional shape that is intended to be non-circular. As used herein, a non-round scope is not meant to include a scope manufactured to have a circular cross section shape that is now damaged and considered non-round. Instead, non-round as used herein refers to an intentional design choice to make the cross section shape of the scope non-circular. FIGS. 15A-15B show the non-round scope 4401 of FIG. 14A without the sheath. As shown in FIGS. 15A-B, the distal face of the scope 4401 is non-round. Instead, the face is substantially triangular with rounded edges. The face further includes a lens 4455 and a lighting component 4456.

As shown in FIG. 14A, the complementary sheath 1422 maintains the circular or round exterior shape such that the scope-sheath combination may be easily used with trocars, introducers, or other surgical systems. As used herein, a round sheath can mean that with a substantially circular exterior cross-section. While described as having a round or circular external shape, the complementary sheath designs described herein are not so limited. The complementary sheath has an interior lumen sized, shaped and dimensioned to engage with the exterior surface shape of the non-round scope. The complementary sheath has an exterior shape selected to cooperate with the other surgical systems—such as introducers, trocars or pressure systems—intended for use with the inventive non-round scope-sheath combination. The sheath exterior shape may be circular, oval, elliptical or other regular geometric shape. Alternatively, the sheath exterior shape may have an irregular shape, multi-sided shape or other shape suited for the intended purpose of the non-round scope-sheath combination.

Freed from the design constraints of the round exterior walls, circular cross section shape and overall cylindrical body style, non-round scopes may be embodied in a wide variety of different shapes, as best seen in FIGS. 15B and 19A-21. As further detailed in the embodiments that follow, non-round scopes may include only the key elements of the lighting and visualization system in a sealed re-usable body style that is easy to clean and simple to quickly sterilize. Working channels, conduits for air, insufflation, irrigation, vacuum, and the like for use during endoscopic or laparoscopic surgery can be provided by the complementary sheath. Since the sheath is disposable, the concerns of cleaning working channels and other difficult to clean areas of conventional endoscopes are not a concern. After completing a procedure with a non-round scope and sheath as described herein, the sheath can be disposed of, and the reusable non-round sheath is cleaned, sterilized and prepared for the next use.

The non-round scope may further take advantage of the shrinking sizes of visualization components such as high definition cameras, fiber optic systems, LED and other lighting systems and the like. The result is to uncouple the design requirements of the scope from being circular or round to permit sealing with other surgical introducers (i.e., trocars, cannulas, and the like).

Referring to FIG. 14B, the endoscopic system can further include a handle, gas inlet, fluid inlet, and video or optical cable connection port, similar to as shown and described with respect to FIGS. 2A-3. The distal end of the non-round scope and complementary sheath combination, in use, provides one or more visual field improvement actions for the visualization element to provide active maintenance of an unimpaired view of the surgical field, such as by flowing gas over the lens.

Referring to FIGS. 14C-14D, the distal end 4423 of the sheath 4422 can be configured to conform with, and extend around and over, the distal face of the scope 4401. The distal end 4423 of the sheath 422 can thus function as a deflector assembly for gas provided through the sheath and/or scope. The deflector assembly projects beyond the distal end of the scope and also a predetermined distance towards the central axis of the scope and the lens 4455. The deflector assembly thus overhangs the distal face of the scope by a prescribed transverse distance sufficient to change the direction of gas flowing axially through conduits into a non-axially, transverse path across the laparoscopic lens. The distance of the deflection width does not extend to the point that is obstructs the field of the view of the laparoscopic lens. The deflector assembly also projects axially beyond the distal terminus of the scope by a prescribed axial distance, defining an air channel or nozzle, similar to as described above with respect to FIG. 6C. The nozzle can have a width of between 0.005 inches and 0.010 inches. The deflector assembly is sized and configured to direct the portion of the air/gas that is conveyed through the conduits in a prescribed flow path and flow velocity across the lens, as will be described in greater detail later.

The deflector assembly (distal end 4423) can further include stand-offs 4412, similar to as described above with respect, for example, to FIGS. 6A-6C. The stand-offs 4412 can be configured to sit against the distal face of the scope and/or to direct gas flows as desired.

In some embodiments, the sheath 4422 can attach to the scope 4401 at the proximal end of the scope. The proximal end 4493 of the non-round scope 4401 is shown more closely in FIGS. 15C and 15D. As shown, the proximal end 4422 can include a cable inlet 4494 around which the sheath 4422 can attach, such as with a fork feature. Such a fork attachment mechanism is described, for example, in U.S. patent application Ser. No. 12/635,632, filed Dec. 10, 2009, titled “VIEW OPTIMIZER AND STABILIZER FOR USE WITH SURGICAL SCOPES,” now U.S. Pat. No. 9,050,037, the entirety of which is incorporated by reference herein. The proximal end 4422 also includes a groove 4495 therein that can additionally or alternatively be used for attachment of the sheath 4422, such as through a locking collar or annular clip. The other recesses, ridges, and bosses shown in FIGS. 15C-15D can likewise be used additionally or alternatively for attachment of the sheath.

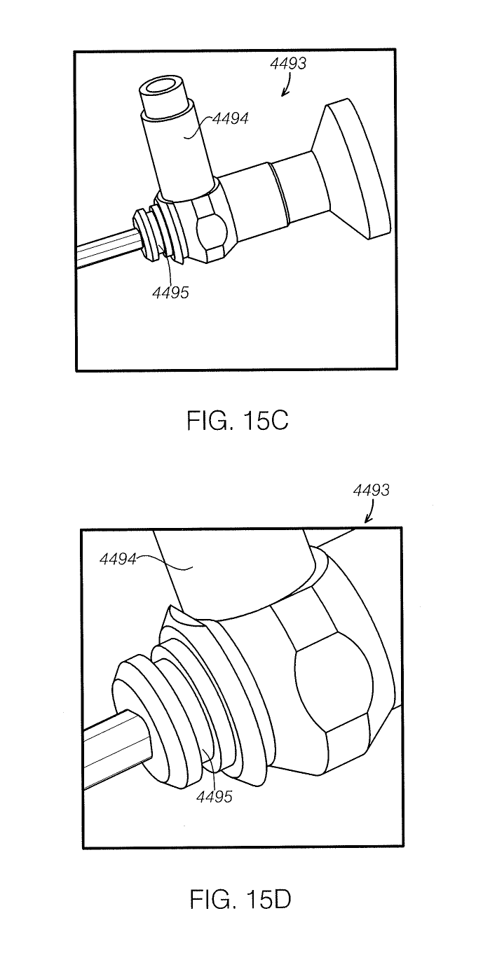

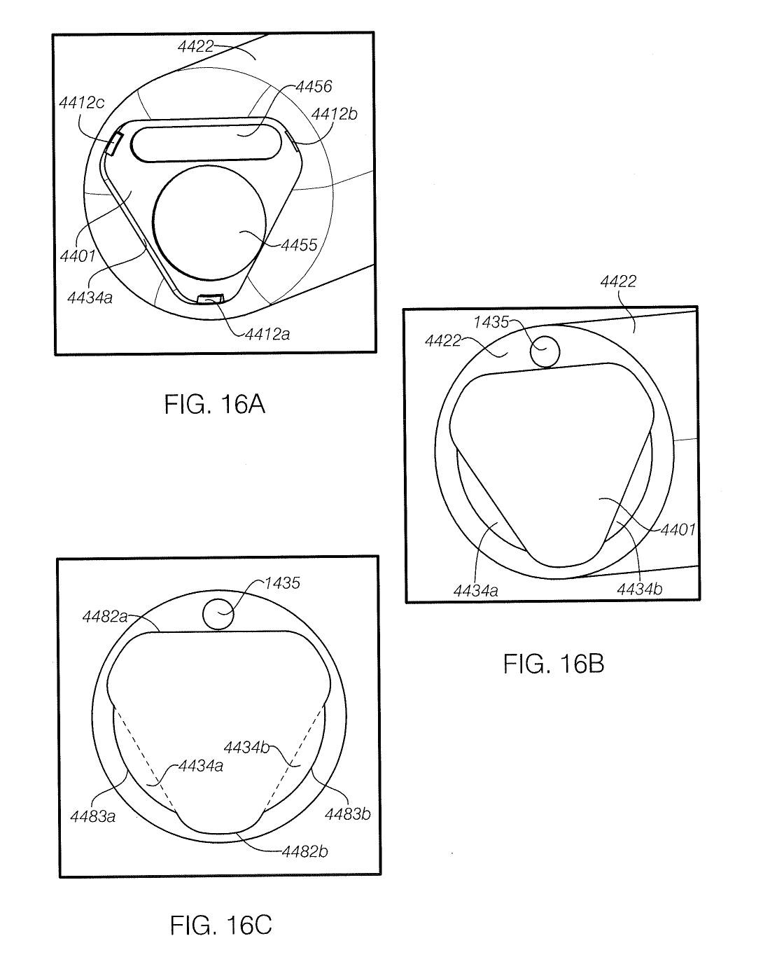

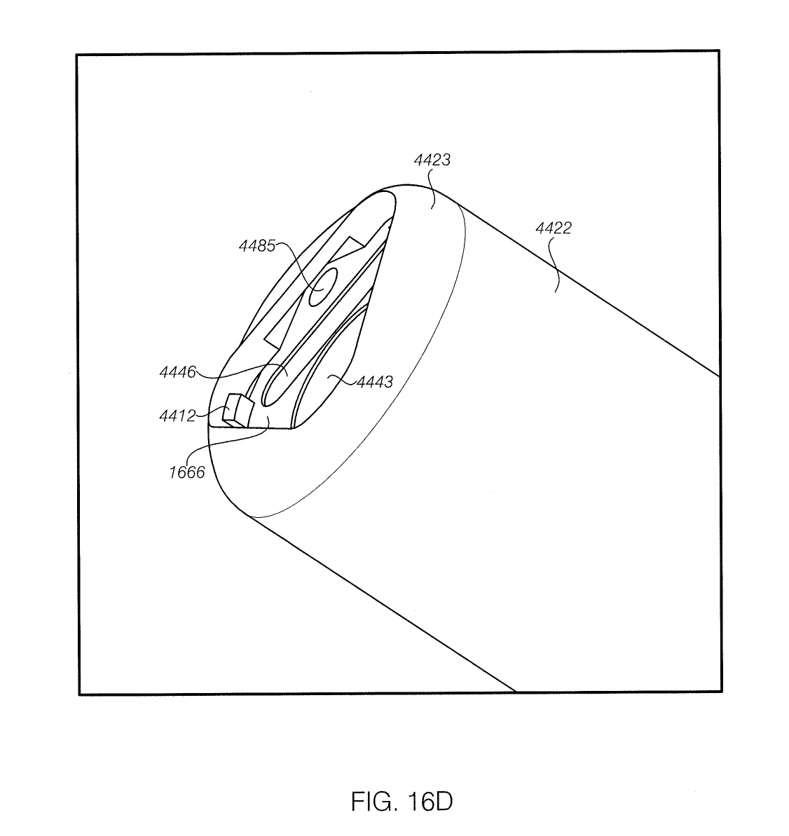

Referring to FIGS. 16A-16C, in some embodiments, the sheath 4422, when placed about the non-round scope 4401, can be configured to form conduits 4434a-b therebetween that can be used, for example, to deliver gas to the distal end of the scope and/or allow working elements to pass therethrough. The conduits 4434a thus have one side formed by the scope external wall and one side formed by the sheath interior wall. As shown in FIG. 16C, the internal perimeter of the sheath 1422 can be shaped so as to have engaging portions 4482a,b configured to closely engage with and/or seal against the outer perimeter of the scope 4401 and to have conduit portions 4483a,b that extend away from the scope to form the conduits 4434a,b.

The sheath 4422 thus has grooves or shaped portions placed around the circumference of the interior sheath wall and running the length of the sheath. Once the non-round endoscope 1401 is placed within this complementary sheath design, the grooves or shaped portions of the sheath interior walls align with the exterior walls of the endoscope to form the conduits 4434a,b of the endoscope-sheath combination.

In one aspect, there is a non-round endoscope having working channels that are formed in part by grooves, cut outs or conduits extending along the length of a non-disposable or re-useable component. When inserted into and engaged with an appropriately configured complementary sheath, the grooves, cut outs, or conduits are covered up by an interior portion of the sheath. After use, the sheath is removed and discarded. Advantageously, the grooves, cut outs, or conduits that were used as working channels can be readily cleaned since they were formed in the external surface of the scope. Wiping down and sterilizing an external surface is a greatly simplified sterilization procedure, unlike the conventional working channels that are positioned on the inside of a scope. In another aspect, one or more channels for gas or CO₂ are be formed when an external sheath is placed over the scope.

Advantageously, using the interior sidewall to accommodate the non-round scope while maintaining a circular outer dimensions allows for a gas seal when the non-round scope-sheath combination is placed through a trocar. In one aspect, the inner portion of the sheath (i.e., that which engages with exterior of the scope) has orientation features that match those of the grooves on the scope. As a result, channels for gas, fluid or passage of instruments or other uses common to the field of endoscopy are formed, by way of illustration only, when: (1) a portion of a channel wall is formed by a portion of an inner wall of the sheath and a portion of the outer wall of the scope, (2) a portion of a channel is formed by a circular inner diameter of a disposable member such as the sheath thus forming the outer wall of the channel, (3) the exterior walls of the grooved outer diameter portion of the non-round scope are used to form the inner portion and remainder of the channel perimeter or circumference, (4) or the disposable member or complementary sheath may have one of more fully contained lumens, channels or conduits configured to fit into one or more of the grooves in the scope external walls. In such configurations, instead of forming part of an active gas conduit, fluid conduit or working channel, a scope groove acts as a registration feature for a conduit or channel provided completely by the sheath.

In some embodiments, the outside of the endoscope includes one or more grooves sized and shaped for various uses as is common in endoscopic procedures and surgery. The grooves extend along the length of the scope and are positioned where needed according to the specific design considerations for a particular scope or procedure. In this embodiment, there are no unsealed internal channels in the endoscope. Instead, an endoscope according to one aspect would have a lighting system and visualization component sealed within the distal portion and scope interior along with the external grooves, placed around the circumference of the scope casing and running the length of the scope external encasement. Once placed within a complementary sheath, the sheath interior walls and the external grooves align to form the working channels and conduits of the endoscope-sheath combination.

Referring to FIGS. 16B-16C, in some embodiments, the sheath 4422 can also include a separate conduit 4435, such as to deliver fluid to the end of the scope and/or over the lens. As shown, in one embodiment, the conduit 4435 can be embedded within the wall of the sheath 4422. Referring to FIG. 16D, in some embodiments, a plate 1666 can be placed between the distal end of the scope and the inside of the deflector 4423. The plate 1666 can be used, for example, to seal the opening 4485 for the fluid conduit to keep it from leaking into other portions of the scope. The plate 1666 can thus include an opening 4443 for the lens, an opening 4446 for the lighting component, and an opening 4485 for the fluid conduit. Advantageously, a plate such as plate 1666 can be used to convert any open channel (such as for gas or working channel) to a fluid channel.

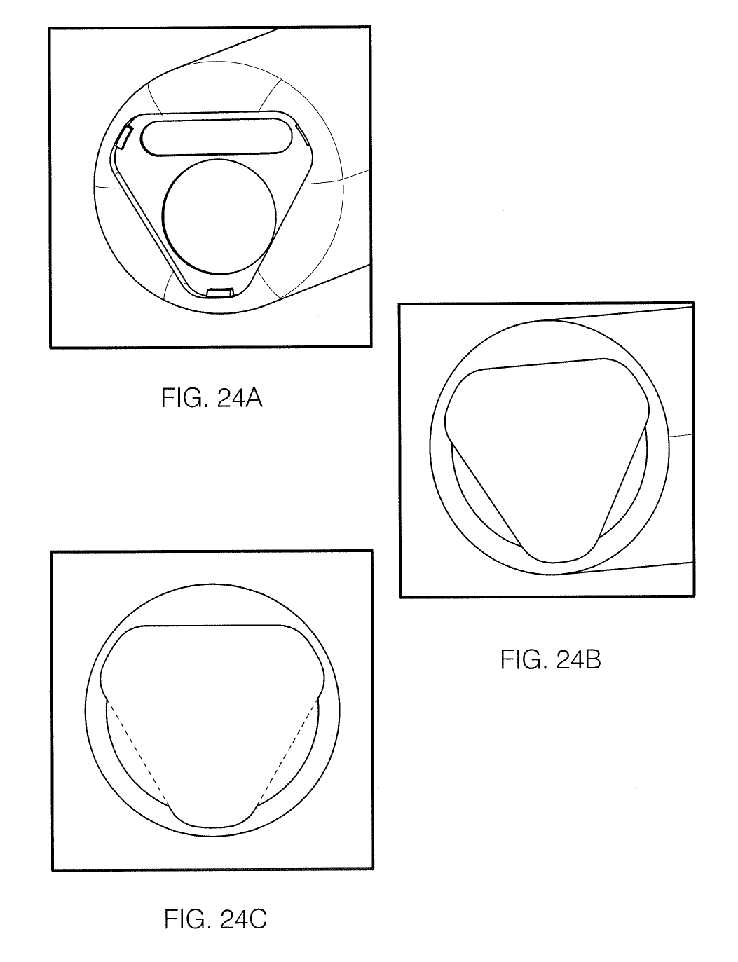

FIGS. 24A, 24B and 24C are similar to those of FIGS. 16A, 16B and 16C with the exception that the optional in-sheath conduit is omitted. FIG. 24A is an isometric view of the distal end of the non-round endoscope and sheath combination of FIG. 14A where the fluid conduit is removed. In one aspect, the dual ports shown in FIG. 14A may be used where each port is aligned to be in communication one of the lumens formed when the non-round scope and sheath are mated for use. Optionally, a single gas line may be used (i.e., one of the ports shown in the embodiment of FIG. 14A is removed) to supply the gas used in the scope to provide the one or more vision improvements. FIG. 24B is cross section view of the isometric view of FIG. 24A taken proximal to the distal end showing the complementary fit of the non-round scope exterior surface and the interior lumen of the sheath. The dedicated within sheath channel of FIG. 16A is removed from the embodiment illustrated in FIG. 24B, leaving only channels having one side formed by the scope external wall and the sheath interior wall. FIG. 24C is cross section view of the view of FIG. 24B with the non-round scope removed. Indicated in this view are portions of the sheath wall shaped to engage with the scope external wall and portions of the sheath wall shaped to form one or more channels. While two channels are shown, more channels, different sized and shaped channels are possible depending upon the desired configuration and surgical use for a specific scope-sheath combination.

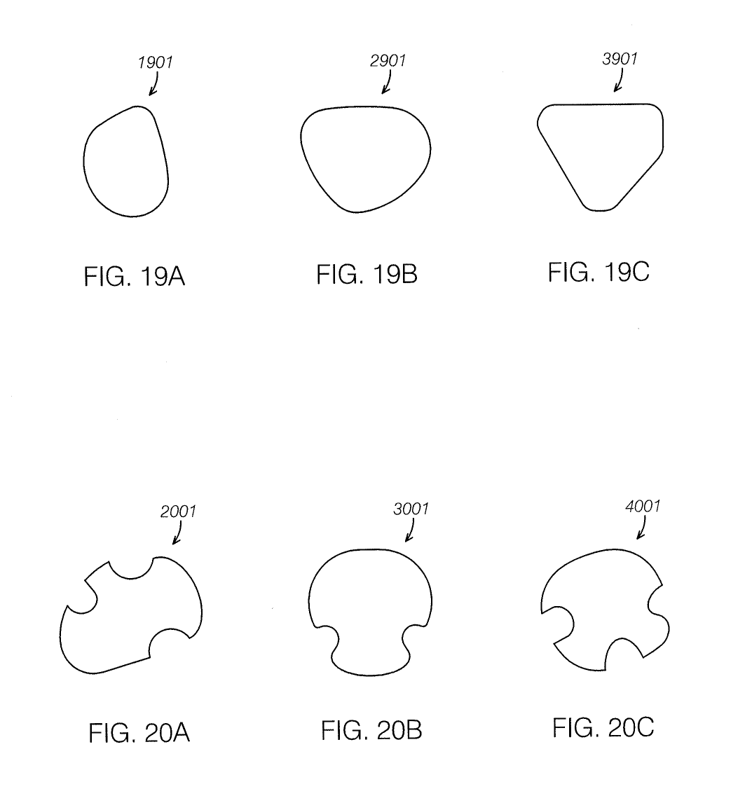

Various additional shapes for non-round endoscopes are shown in FIGS. 19A-20C. FIG. 19A is an exemplary non-round scope 1901 with a cross-section having a partially flattened ovoid or elliptical shape. FIG. 19B is an exemplary non-round scope 2901 with a cross section having a D-shape. FIG. 19C is an exemplary non-round scope 3901 with a cross section having a generally triangular shape similar to that of FIG. 15B with at least one corner flattened. FIG. 20A is an exemplary non-round scope 2001 with a cross section having two cut outs that form conduits when the scope is inserted into a complementary sheath. FIG. 20B is an exemplary non-round scope 3001 with a cross section having three cut outs that form conduits when the scope is inserted into a complementary sheath. FIG. 20C is an exemplary non-round scope 4001 with a cross section having three cut outs that form conduits when the scope is inserted into a complementary sheath.

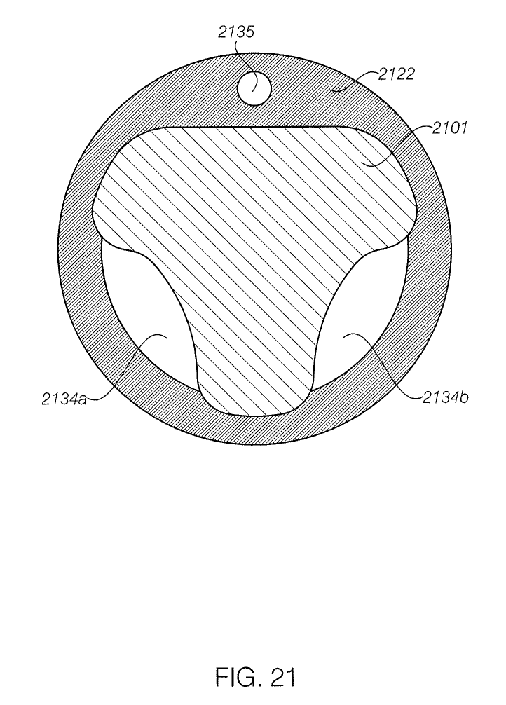

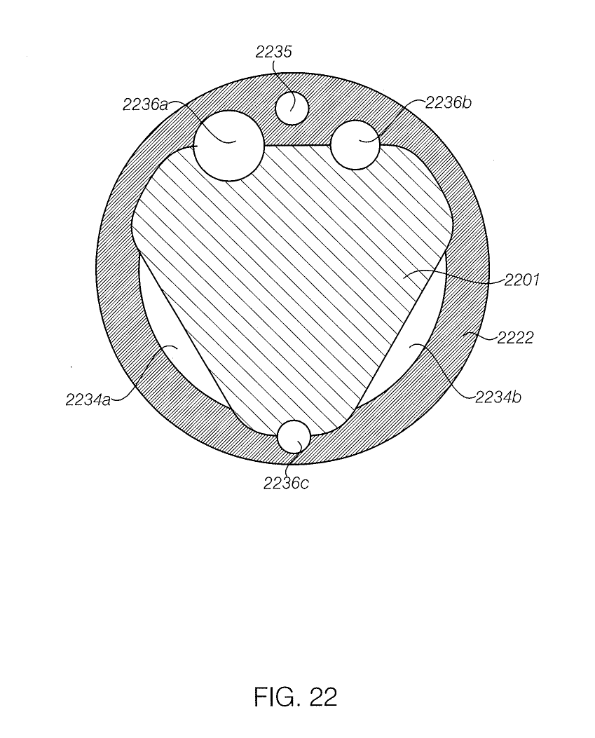

Any of these non-round scopes can be used with a sheath as described herein that can form conduits when engaged therewith and/or that make the cross-section of the combined scope/sheath substantially round. For example, FIG. 21 is a section view of a non-round scope 2101 and sheath 2122 combination showing the formation of channels 2134 along the scope bounded by an interior wall of the sheath 2122 and a shaped portion of the exterior wall of the scope 2101. A channel 2135 completely within the sheath is also shown. Another exemplary non-round scope 2201 and sheath 2222 combination is shown in FIG. 22. In addition to conduits 2234a,b and channel 2235, channels 2236a,b,c are also formed by the alignment of cooperatively shaped portions of the non-round scope 2201 external wall and the sheath 2222 interior wall.

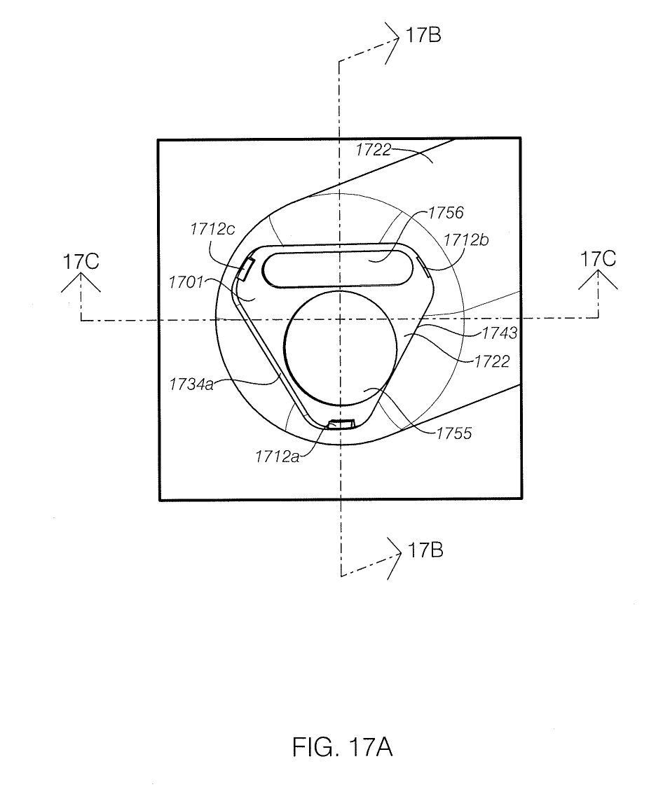

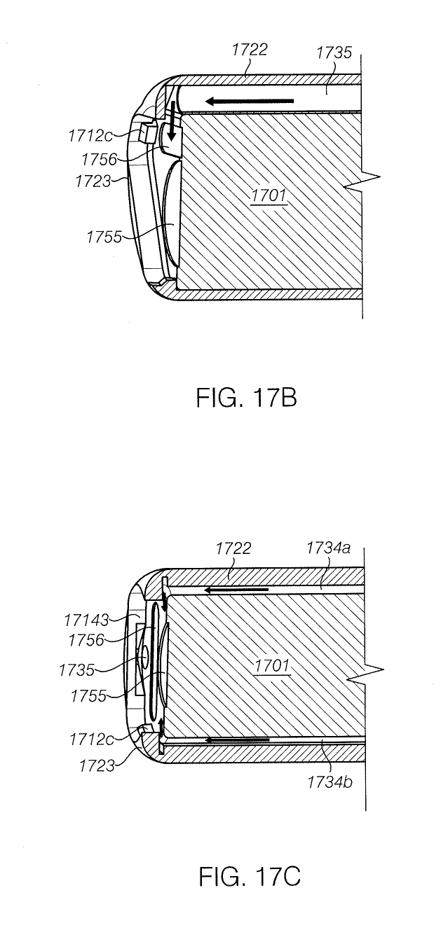

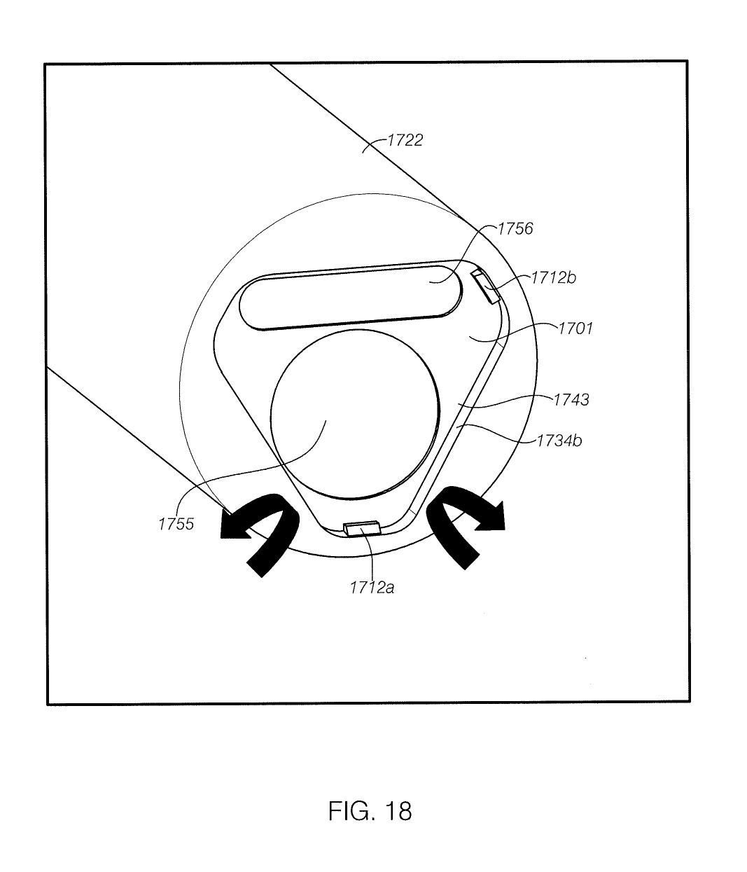

Referring to FIGS. 17A-18, in one embodiment, the sheath and scope can together be used to form conduits that can be used for defogging and cleaning the scope. That is, referring to FIG. 17C, gas (shown by the arrows) can flow down conduits 1734a,b formed between the outer perimeter of the scope 1701 and the inner perimeter of the sheath 1722. When the gas reaches the end of the scope 1722, it can hit the deflector assembly 1723 and be channeled (through stand-offs 1712a,b,c) towards the hole 1743 in the sheath 1722 and thus towards the lens 1755 and lighting element 1756. In some embodiments, the velocity of gas through the conduits 1734a,b can be tailored such that one is higher than the other so as to form a vortex over the distal end of the scope and/or over or proximate to the lens, as is described further below. The vortex airflow and/or gas can be on the left and/or right side of the lens (as shown by the arrows in FIG. 18). The vortex can be formed such that it extends over a peripheral part of the lens and/or off to the side of the lens (i.e., not right along the central axis) so that it keeps the field of view clear (i.e., without creating a swirling affect right in the center of the resulting image). In some embodiments, the vortex is created in an upper portion of the lens, a lower portion of the lens, and/or a side portion of the lens.

Further, referring to FIG. 17B, in use, fluid can be supplied (as shown by the arrows) through the in-sheath conduit 1735 so as to provide additional cleaning of the lens where necessary. The fluid can exit close to the lighting component 1756 and be directed across the lens after hitting the deflector assembly 1723 and being directed by stand-offs 1712b,c.

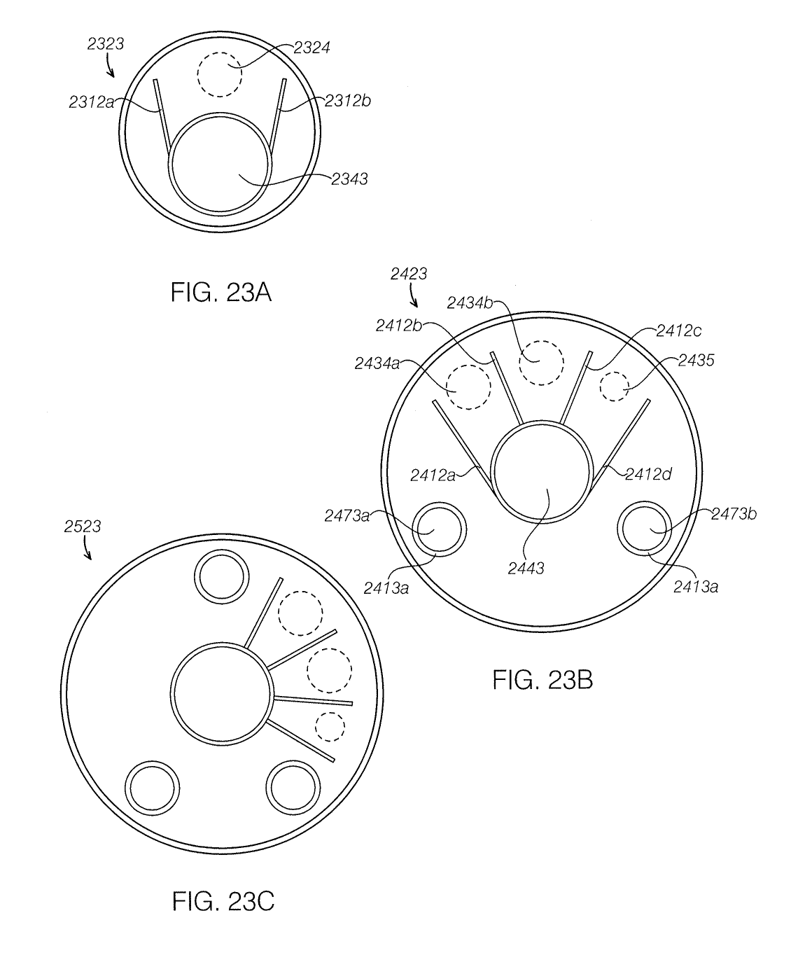

FIGS. 23A-23C show different embodiments of the interior of a deflector assembly 2323a with relative placement of gas or fluid conduits (formed within the sheath and/or between the sheath and the scope) shown in dotted lines. The deflector assembly 2323 includes stand-offs 2312a and 2312b that extend radially from the opening 2343 (for the lens) and on either side of the gas outlet 2334 to direct gas towards the opening 2343.

FIG. 23B shows a deflector assembly 2423 configured so as to attach to a scope and cover substantially all of the tip face with the exception of the lens (via the opening 2443) and the working channel (via working channel openings 2473a,b). Stand-offs 2412a,b,c,d extend radially from the opening 2443, separating the outlets gas 2434a,b and fluid outlet 2435, to direct gas and/or fluid towards the opening 2443. Additional stand-offs 2413a,b are provided around the working channel openings 2473a,b to seal the working channel (for allowing instruments to pass therethrough) and to maintain gas integrity within the deflector assembly.

FIG. 23C shows a deflector assembly 2523 that is the same as deflector assembly 2423 shown in FIG. 23B, but includes an additional working channel opening.

A number of gas diverters or stand offs are illustrated in the various views of FIGS. 14C, 3A, and 23A-23C. The gas diverters redirect gas or fluids introduced into the complementary sheath towards openings adjacent to an area where a vortex is to be formed. In one embodiment, the openings used to create a vortex may be provide by one or more stand offs inside of the complementary sheath. The fit between the complementary sheath and the tip face/tip engagement region (depending on design) on the non-round endoscope is adapted and configured to prevent gas loss. As a result, most of the gas or fluid introduced into the complementary sheath via the one or more gas inlets (see FIG. 14B) is directed through the gas channels to the gas openings in relation to the lens opening to form a vortex with the desired properties. In one alternative aspect, the complementary sheath or tip face may be modified to enhance the gas tight seal along the gas diverters and/or the standoffs in order to reduce or minimize loss of gas or fluid through the gas channels.

There are a number of advantages to providing visual field improvement to a re-usable non-round cross section endoscope, especially when configured as an endoscope or laparoscope, since such instruments can be readily decontaminated and re-sterilized. As with conventional scopes, the non-round scopes described herein may be made in different lengths depending upon application or surgical need. Similarly, the distal end of the non-round scopes can be made in different angles (0, 30, 45 degrees) and in different diameters (5 mm, 10 mm). In each case, the corresponding sheath distal end, gas and fluid conduits, and stand offs are also adapted and configured for use with the associated camera angle and non-round scope shape and dimensions.

Still further, the complementary sheath may be disposable as well as designed to accommodate the combinations of sizes and angles as well as working channels or other ports, depending upon the design characteristics of a particular non-round endoscope. In each of these different configurations, the complementary shaped sheath has a distal portion that is adapted and configured direct the gas around the endoscope tip (i.e., in proximity to one or more visualization components of the scope) from a single gas supply lumen or from multiple gas supply lumen in communication with the sheath.

The scopes, tip caps, and/or sheaths described herein can be configured to provide one or more visual field improvement actions during imaging with the scope. In one aspect, a visual field improvement action is one or more of: a gas flow pattern relative to the visualization component to remove condensation therefrom, a gas flow pattern relative to the visualization component to form an air barrier to reduce or minimize particles in the visual field of the visualization component and a gas flow pattern relative to the visualization component to facilitate removal of a fluid applied to the visualization component.

To achieve the improved visualization, the scope, tip caps, and/or sheaths can include one or more gas and/or fluid channels for providing a vortex of gas/air and/or a cleaning fluid across the lens as described in the '084 application.

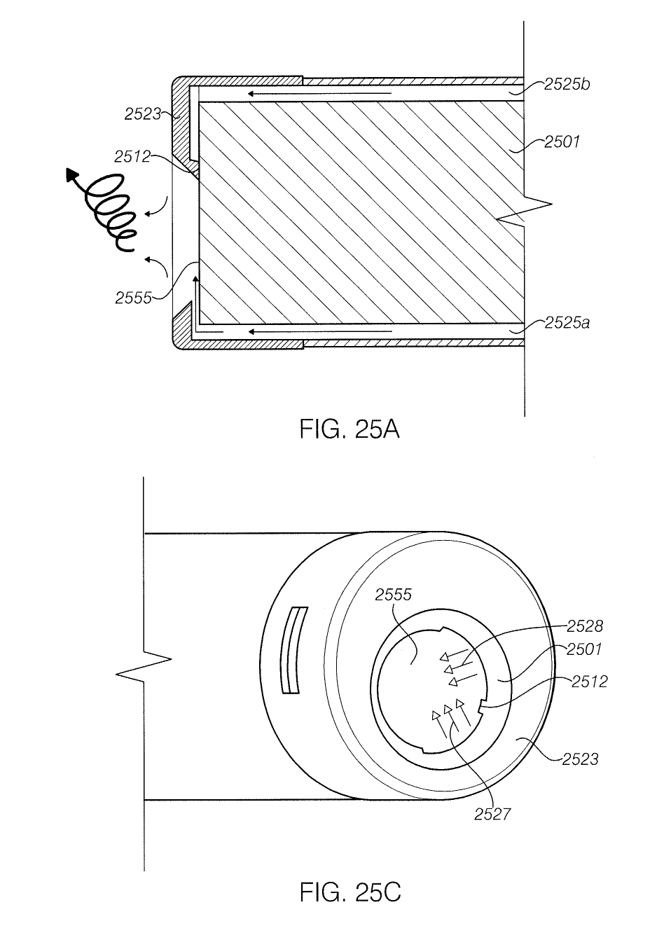

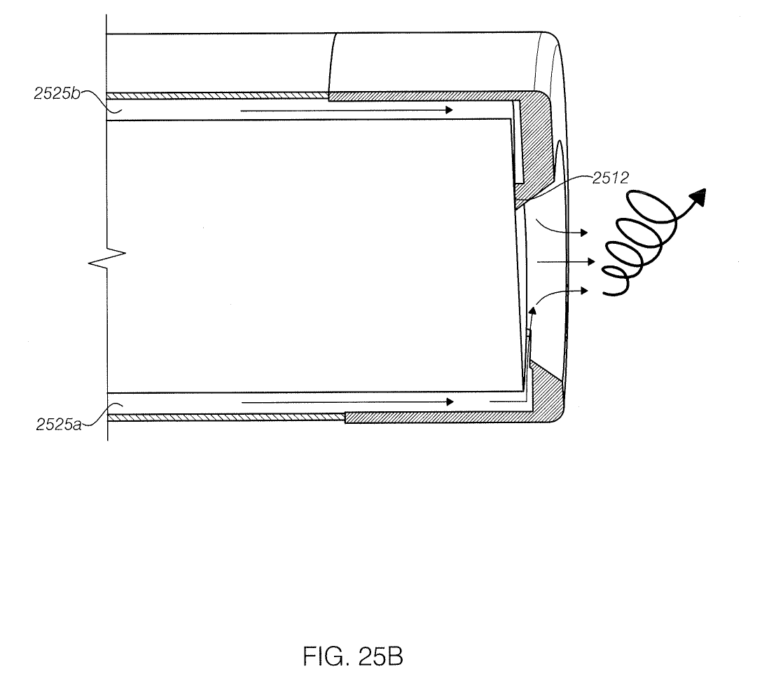

FIGS. 25A and 25B shows the exemplary formation of a vortex of gas over or proximate to the lens 2555 of scope 2501. In this embodiment, gas can travel distally through gas conduits 2525a,b (formed either through a sheath, through the scope, or between the sheath and scope), hit a deflector assembly 2523 (which can be at the distal end of a sheath or part of a tip cap), and form a vortex proximate to the lens 2555. As shown in FIG. 25C, the vortex can be formed by having a first gas flow 2528 that is of higher velocity than a second gas flow 2527. A divider or stand-off 2512 can separate the first gas flow 2528 from the second gas flow 2527. The gas flow 2528 can have a higher velocity airflow than the gas flow 2527, for example, by creating cut-outs at a proximal end of the sheath as described in the '084 application, by placing a restrictions in the gas conduit supplying gas flow 2527 at the proximal end, or by placing a restriction in the gas conduit supplying gas flow 2527 at the distal end near the deflector. When the high and low velocity gas flows 2528, 2527 meet, they can combine to form a vortex (as shown in FIGS. 25A and 25B).

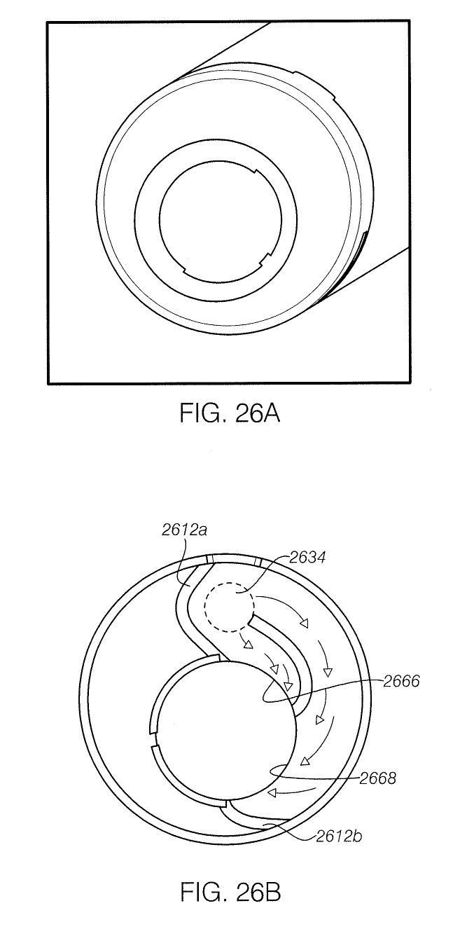

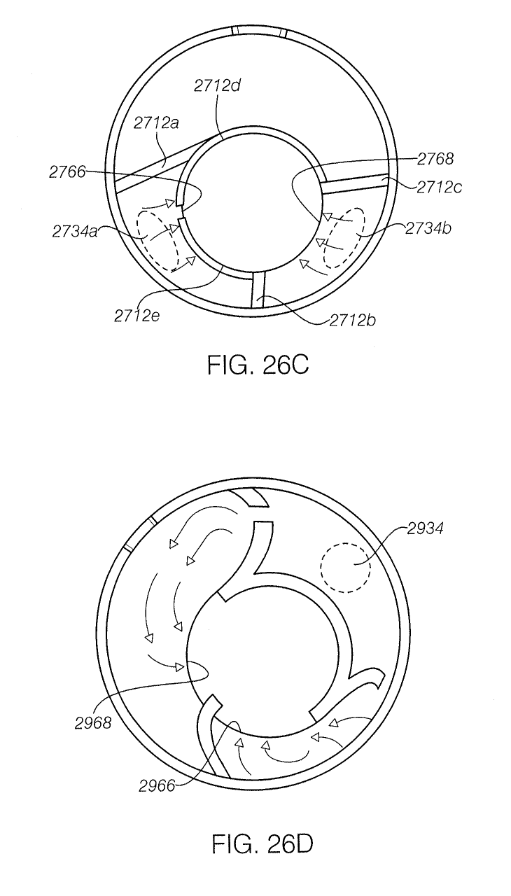

FIGS. 26A-26D show various embodiments of an exemplary scope 2601 with a deflector assembly 2623 thereon (which can be the distal end of a sheath or part of a tip cap, as described herein) that can be used to form a vortex over or proximate to the lens.

Referring to 26B, in one embodiment, the deflector assembly 2623 can include stand-offs 2612a,b,c that serve to direct the flow of gas (shown by the arrows) from a single gas lumen 2634. Because nozzle opening 2666 is closer to the gas lumen 2634 and/or because the nozzle opening is smaller in diameter (i.e., has a greater restriction), the flow of gas through opening 2666 can have a higher velocity than the flow of gas through opening 2668, thereby providing for the formation of a vortex.

Referring to FIG. 26C, in another embodiment, the deflector assembly 2623 can include stand-offs 2712a,b,c that separate gas flow from two different lumens 2734a,b. Because nozzle opening 2766 is restricted by stand-offs 2712d,e, it can produce a higher velocity of air flow therethrough than nozzle opening 2768, thereby providing for the formation of a vortex.

In embodiments where the difference in velocity changed by a restriction in the opening of a higher velocity nozzle relative to a lower velocity nozzle, the nozzle with the larger restriction (i.e., the smaller diameter, length, perimeter, cross section, and/or width) will produce the higher velocity.

In some embodiments, where various gas conduits are used down the length of the scope, a plenum section can be provided within the distal tip (sheath or tip cap) that allows air from each of the conduits to combine into a single velocity airflow before entering the nozzles. This plenum section can be configured similarly to the plenum described in the '084 application. FIG. 26D shows an exemplary position of the outlet 2699 of the plenum section relative to the two gas flows through nozzles 2966, 2968. The position of the plenum outlet 2599 can be adjusted and/or the nozzle opening size can be adjusted, as described herein, so as to adjust the velocity of the two airflows and provide for the formation of a vortex.

As described above, a single velocity flow (either from a gas conduit or a plenum) can thus be divided up between two or more flows of differing velocities to form the desired vortex.

In some embodiments, the gas is provided by a gas supply, insufflator, or recirculating gas system, as described in the '084 application. As used herein, the terms “air,” “gas,” “CO₂”, or “surgical gas” can be used interchangeably.

In some alternatives, the tip cap, sheath, and/or scope may be modified as need to provide one or more visual field improvement actions to any of the following endoscope, gas line and fluid line combinations I to VIII:

| Configuration | Gas Lines | Fluid Lines |
|---|---|---|
| I | 1 | 0 |
| II | 1 | 1 |
| III | 2 | 0 |
| IV | 2 | 1 |
| V | 3 | 0 |
| VI | 3 | 1 |
| VII | 4 | 0 |
| VIII | 4 | 1 |

It is to be appreciated that the features of any embodiment of the scope, sheath, and/or tip cap described herein can be combined with any other embodiment. Likewise, any features may be subtracted and/or added from each of the embodiments.

Various aspects of gas and fluid supply lines, scope, complementary sheath and/or tip may be provided to correspond to the various alternative environments described in the applications and patents that follow. As such, endoscopes, sheaths, and/or tip caps—including those that work with rigid, semi-rigid and flexible systems—may be modified, adapted and configured as described herein to adjust interior lumen space allocation, provide for the inclusion of one or more gas and/or fluid conduits, include gas manifolds along with various complementary sheaths, and/or have scope exterior walls/sheath interior wall engagement regions, each of which may be modified or adapted for providing visual field improvement actions to numerous different surgical or operating environment or tools used in those environments or procedures. Still further improvements to visualization components, lighting systems, camera, optical sensors and the like are described in the following, each of which is incorporated by reference in its entirety:

Non-circular shaped endoscopes having more than one visualization component on different axis or orientation and other aspects are described in U.S. Patent Application Publication No. 2013/0172670 to Levy et al., titled “REMOVABLE TIP ENDOSCOPE,” filed Dec. 13, 2012 as well as in U.S. Patent Application Publication No. 2013/0317295 to Morse, titled “LIGHT ASSEMBLY FOR REMOTE VISUAL INSPECTION APPARATUS,” filed Dec. 29, 2006.

Endoscopes with various different working channels and interior lumen utilization designs and other aspects that may be incorporated into the scopes herein are described in U.S. Pat. No. 8,517,921 to Tremaglio et al., titled “ENDOSCOPIC INSTRUMENT HAVING REDUCED DIAMETER FLEXIBLE SHAFT,” filed Apr. 18, 2005.

Optional, additional details for variations to the scope and sheath/cap tip systems described herein may be obtained by reference to FIGS. 20, 6 and 7, respectively, of U.S. Pat. No. 6,206,825 to Tsuyuki, titled “ILLUMINATION SYSTEM FOR ENDOSCOPES AND AN ENDOSCOPE HAVING THE ILLUMINATION SYSTEM,” filed Jan. 15, 1999, incorporated herein by reference.

Endoscopes adapted for use with flexible, multiple scope or also various robotic applications and other aspects that may be incorporated into the scopes herein are described in U.S. Patent Application Publication No. 2010/0331856 to Carlson et al., titled “MULTIPLE FLEXIBLE AND STEERABLE ELONGATE INSTRUMENTS FOR MINIMALLY INVASIVE OPERATIONS,” filed Dec. 14, 2009. Various aspects of gas and fluid supply lines, complementary sheath and other alternatives may be provided to correspond to the various alternative environments of, for example, FIG. 4A-4K, 7A, 8, or 9. Various aspects of complementary sheath/cap tip and alternatives may be provided to correspond to the various moveable, multiple or split imaging device alternative environments of, for example, FIGS. 5A, 5B and 6.

Additional robotic applications and systems are described in U.S. Patent Application Publication No. US 2014/0371763 to Poll, et al., titled “SHEATH FOR HAND-HELD AND ROBOTIC LAPAROSCOPES,” published on Dec. 18, 2014. Various aspects of gas and fluid supply lines, complementary sheath and scope aspects and alternatives may be provided to correspond to the various alternative environments of, for example, FIGS. 3, 4, 5A-8B particularly for multiple cameras, angled tips as in FIGS. 9B-1013. In one aspect, the robotically controlled laparoscope (see for example FIGS. 11, 18, 19, 20, 22 and 23) is modified to accommodate the innovative modifications of the scope—sheath combination to provide the one or more gas conduits, fluid conduits, scope wall-sheath wall engagement regions and complementary sheath designs to enable the advantageous use of one or more visual field improvement actions during robotic surgery. Various aspects of complementary sheath/tip cap and scope alternatives may be provided to correspond to the various embodiments described as well as for use with other robotically controlled surgery systems, such as, for example, the Da Vinci system available from Intuitive Surgical, Inc.

In still other aspects, flexible multiple segment endoscopes are described in U.S. Pat. No. 7,087,013 to Belson et al., titled “STEERABLE SEGMENTED ENDOSCOPE AND METHOD OF INSERTION,” issued Aug. 8, 2006. Various aspects of gas and fluid supply lines, working channels and other aspects of the complementary sheath/tip cap and scope alternatives may be provided to correspond to the various alternative environments of, for example, FIGS. 2, 3, 4, 7-11B, and 21 as well as for providing one or more visual field improvement actions adapted for the methods corresponding to FIGS. 12-20 and 24-26.

In still other aspects, flexible multiple segment, tendon driven endoscopes are described in U.S. Pat. No. 6,858,005 to Ohline et al., titled “TENDON-DRIVEN ENDOSCOPE AND METHODS OF INSERTION,” issued Feb. 22, 2005. Various aspects of gas and fluid supply lines, working channels and other aspects of the complementary sheath and scope alternatives may be provided to correspond to the various alternative environments of, for example, FIGS. 3A-5, 6D, 6E, 7A, 7B, as well as for providing one or more visual field improvement actions adapted for the methods corresponding to FIGS. 12A-12F and 13.

Various aspects of gas and fluid supply lines, working channels and other aspects of the complementary sheath and scope alternatives may be modified or provided to correspond to include one or more aspects of the designs in U.S. Patent Application Publication No. 2015/0038785 to Govrin et al., titled “INTEGRATED ENDOSCOPE IRRIGATION,” filed Sep. 15, 2014, modified for use in providing one or more visual field improvement actions.

Various aspects of gas and fluid supply lines, working channels and other aspects of the complementary sheath and scope alternatives may be provided to correspond to the various endoscopes of various sizes and alternative configurations—including other aspects as described in U.S. Pat. No. 5,857,961 to Vanden Hoek et al., titled “SURGICAL INSTRUMENT FOR USE WITH A VIEWING SYSTEM,” filed Feb. 6, 1996.

Additional details and alternatives for visualization components for use in the herein described scope—sheath combinations may be provided in U.S. Patent Application Publication No. 2007/0182842 to Sonnenschein et al., titled “REUSABLE MINIATURE CAMERA HEAD,” filed Nov. 27, 2006; PCT Publication No. WO2005/002210 to Sonnenschein et al., titled “AUTOCLAVABLE IMAGER ASSEMBLY,” filed Jul. 31, 2003; EP Publication No. EP 0 790 652 to Sano et al., titled “SOLID-STATE IMAGE PICKUP DEVICE AND ITS MANUFACTURE,” filed Jul. 30, 1996; PCT Publication No. WO2005/115221 to Sonnenschein et al., titled “A REUSABLE MINIATURE CAMERA HEAD,” filed May 30, 2005.

In still further aspects, the visualization component comprises a lens system and a solid state sensor. In some embodiments, the solid-state sensor is selected from the following group: a Charge Coupled Device (CCD); an Intensified Charge Coupled Device (ICCD); an Electron Multiplying Charge Coupled Device (EMCCD); and a Complementary Metal Oxide Semiconductor (CMOS) device. In some embodiments, the visualization device includes a sensor as in claim 42 having a diagonal size in the range from approximately 0.5 mm, 1 mm, 1.5 mm, 2 mm, 2.5 mm, 3 mm, or 3.5 mm, or 4 mm.

In some embodiments, the scope includes a visualization device with a lens system having a plurality of lens that together form an image with a field of view of between 60 and 140 degrees. In still further alternatives and variations, the scope-sheath combination systems described herein are adapted such that the scope, complementary sheath and visualization device are adapted and configured for carrying out a procedure selected from the following: (1) a gastroscopy procedure by forming an image with a field of view of 120 to 140 degrees; (2) an ERCP procedure by forming an image with a field of view of the camera head of the invention 120 to 140 degrees in the motherscope and by forming an image with a field of view of 100 degrees in the baby scope; (3) a colonoscopy procedure by forming an image with a field of view of 120 to 140 degrees; (4) a gynecology procedure by forming an image with a field of view of 100 to 120 degrees; (5) a bronchoscopy procedure by forming an image with a field of view of 80 to 100 degrees; (6) an ENT procedure by forming an image with a field of view of 80 to 100 degrees; and (7) a transgastric procedure by forming an image with a field of view of 120 to 140 degrees in the motherscope and by forming an image with a field of view of 100 to 120 degrees in the baby scope.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A scope, comprising:
an elongate body having a proximal end and a distal end;
a lens at the distal end of the elongate body;
at least one conduit extending from the proximal end to the distal end configured to connect to an gas supply; and
a view optimizing assembly extending from the distal end of the elongate body past the lens, the view optimizing assembly including:
a first lumen and a second lumen, the first and second lumens in fluid communication with the at least one conduit and configured such that a single velocity flow from the at least one conduit separates into a first flow through the first lumen and a second flow through the second lumen, the first flow having a higher velocity than the second flow;
a plurality of dividers separating the lumens; and
a deflector assembly configured such that gas exiting the first and second lumens combines to keep debris off of the lens.

2. The scope of claim 1, wherein the at least one conduit extends within the elongate body.

3. The scope of claim 1 or 2, further comprising a sheath extending around the elongate body configured to support the view optimizing assembly.

4. The scope of claim 3, wherein the at least one conduit extends between an outer circumference of the elongate body and an inner circumference of the sheath.

5. The scope of claim 3, wherein the at least one conduit extends within the sheath.

6. The scope of any of claims 1-5, wherein the at least one conduit comprises a plurality of conduits.

7. The scope of claim 6, wherein the deflector assembly further comprises a plenum section configured to allow gas from the plurality of conduits to combine into a single velocity gas flow before entering the first and second lumens.

8. The scope of any of claims 1-7, wherein the plurality of dividers comprise a plurality of stand-offs configured to touch a surface of the lens.

9. The scope of claim 8, wherein the at least one conduit comprises a plurality of conduits, and wherein the stand-offs extend from a wall between the conduits.

10. The scope of claim 8, wherein the at least one conduit comprises a single conduit, and wherein the stand-offs divide the gas into the first and second lumens.

11. The scope of any of claims 1-10, wherein the deflector, the distal end of the elongate body, and the dividers together form a first nozzle in communication with the first lumen and a second nozzle in communication with the second lumen.

12. The scope of any of claims 1-11, wherein a length of each lumen is between 0.005 inches and 0.010 inches.

13. The scope of any of claims 1-12, wherein the gas exiting the first and second lumens combines to form a vortex to keep debris off of the lens.

14. The scope of any of claims 1-13, wherein the elongate body is flexible along at least a portion of a length of the elongate body.

15. The scope of any of claims 1-13, wherein the elongate body is rigid.

16. The scope of any of claims 1-13, wherein the view optimizing assembly is attached to the elongate body with a locking mechanism.

17. The scope of any of claims 1-16, wherein the view optimizing assembly is integral with the elongate body.

18. The scope of any of claims 1-16, wherein the first lumen is smaller than the second lumen such that the first flow has a higher velocity than the second flow.

19. A view optimizing assembly for a scope, comprising:
an elongate body configured to extend from a distal end of a scope past a lens of the scope;
a first lumen and a second lumen within the elongate body, the first and second lumens in fluid communication with at least one conduit of a scope and configured such that a single velocity flow from the at least one conduit separates into a first flow through the first lumen and a second flow through the second lumen, the first flow having a higher velocity than the second flow;
a plurality of dividers separating the lumens; and
a deflector assembly configured such that gas exiting the first and second lumens combines to keep debris off of the lens.

20. The view optimizing assembly of claim 19, wherein the at least one conduit comprises a plurality of conduits.

21. The view optimizing assembly of claim 20, wherein the deflector assembly further comprises a plenum section configured to allow gas from the plurality of conduits to combine into a single velocity gas flow before entering the first and second lumens.

22. The scope of any of claims 19-21, wherein the plurality of dividers comprise a plurality of stand-offs configured to touch a surface of the lens.

23. The scope of any of claims 19-22, wherein a length of each lumen is between 0.005 inches and 0.010 inches.

24. The scope of any of claims 19-23, wherein the gas exiting the first and second lumens combines to form a vortex to keep debris off of the lens.

25. The scope of any of claims 19-24, wherein the view optimizing assembly is configured to attach to the scope with a locking mechanism.

26. The scope of any of claims 19-25, wherein the first lumen is smaller than the second lumen such that the first flow has a higher velocity than the second flow.

27. A scope, comprising:
an elongate body having a proximal end and a distal end, the distal end including a tip engagement region;
an interior lumen within the elongate body extending from the proximal end to the distal end;
a tip face adjacent to the tip engagement region and covering the interior lumen;
a gas conduit within the elongate body lumen having an outlet in the tip face and an inlet at the proximal end of the elongate body;
a visualization component in the tip face; and
a tip cap configured to releasably couple with the tip engagement region, wherein the tip cap includes an opening sized for use with the visualization component and at least one stand-off, further wherein, when the tip cap is coupled to the tip engagement region, the opening is positioned around the visualization component and the one or more stand offs engage a portion of the tip face such that a gas flow from the outlet is directed towards the opening to improve viewing through the visualization component.

28. The scope of claim 27, configured such that viewing through the visualization component is improved by one or more of: a gas flow pattern relative to the visualization component to remove condensation therefrom, a gas flow pattern relative to the visualization component to form an gas barrier to reduce or minimize particles in the visual field of the visualization component and a gas flow pattern relative to the visualization component to facilitate removal of a fluid applied to the visualization component.

29. The scope of claim 27, further comprising a visualization component cable connected to the visualization component and in communication with the proximal end of the elongate body.

30. The scope of claim 27, wherein an overall dimension of the tip engagement region is less than the overall dimension of the elongate body proximal portion.

31. The scope of claim 27, wherein an overall dimension of the tip engagement region when coupled to the tip cap is more than the overall dimension of an elongate body proximal portion.

32. The scope of claim 27, wherein an overall dimension of the tip engagement region when coupled to the tip cap is about the same as an overall dimension of an elongate body proximal portion.

33. The scope of claim 27, wherein the tip cap is configured to releasably couple with the tip engagement region using complementary elastic snap fit features.

34. The scope of claim 27, wherein the tip cap is configured to releasably couple with the tip engagement region using a threaded connection.

35. The scope of claim 27, further comprising a handle on the elongate body proximal end supporting the gas conduit inlet and a visualization component cable.

36. The scope of claim 27, further comprising a liquid conduit within the elongate body lumen having a liquid outlet in the tip face and an inlet at the proximal end of the elongate body.

37. The scope of claim 36, the tip cap further comprising one or more liquid stand offs positioned such that, when the tip cap is coupled to the tip engagement region, the one or more liquid stand offs are configured to engage a portion of the tip face such that a liquid flow from the liquid outlet is directed towards the opening to further improve viewing through the visualization component.

38. The scope of claim 37, further comprising a handle on the elongate body proximal end supporting the gas conduit inlet, the liquid conduit inlet, and a visualization component cable.

39. The scope of any of claims 27-38, wherein the elongate body is rigid, semi-rigid or flexible.

40. The scope of claim 39, wherein the elongate body is flexible or semi-rigid, and wherein the scope further includes a handle including a steering mechanism for controlling a bend angle of the elongate body.

41. A scope, comprising:
an elongate body having a proximal end and a distal end, the distal end including a tip engagement region;
an interior lumen within the elongate body extending from the proximal end to the distal end;
a tip face adjacent to the tip engagement region and covering the interior lumen distal end;
a first gas conduit and a second gas conduit;
a visualization component in the tip face; and
a tip cap configured to releasably couple with the tip engagement region, wherein the tip cap includes an opening sized for use with the visualization component and at least one stand-off, further wherein, when the tip cap is coupled to the tip engagement region the opening is around the visualization component and the one or more stand offs engage a portion of the tip face such that the gas flows from the first and second gas conduits towards the opening to improve viewing through the visualization component.

42. The scope of claim 41, wherein the first and second gas conduits are within the elongate body.

43. The scope of claim 41, further comprising a gas inlet and a manifold, wherein the gas inlet is in communication with the manifold, and the manifold is in communication with the first and second gas conduits.

44. The scope of claim 41, configured such that viewing through the visualization component is improved by one or more of: a gas flow pattern relative to the visualization component to remove condensation therefrom, a gas flow pattern relative to the visualization component to form an gas barrier to reduce or minimize particles in the visual field of the visualization component and a gas flow pattern relative to the visualization component to facilitate removal of a fluid applied to the visualization component.

45. The scope of claim 41, further comprising a visualization component cable connected to the visualization component and in communication with the proximal end of the elongate body.

46. The scope of claim 41, wherein an overall dimension of the tip engagement region is less than an overall dimension of a proximal portion of the elongate body.

47. The scope of claim 41, wherein an overall dimension of the tip engagement region when coupled to the tip cap is more than an overall dimension of an elongate body proximal portion.

48. The scope of claim 41, wherein an overall dimension of the tip engagement region when coupled to the tip cap is about the same as an overall dimension of the elongate body proximal portion.

49. The scope of claim 41, wherein the tip cap is configured to releasably couple with the tip engagement region using a complementary elastic snap fit features.

50. The scope of claim 41, wherein the tip cap is configured to releasably couple with the tip engagement region using a threaded connection.

51. The scope of claim 41, further comprising a handle on the elongate body proximal end supporting the first and the second gas conduits and a visualization component cable.

52. The scope of claim 41, further comprising a liquid conduit within the elongate body lumen having a liquid outlet in the tip face and an inlet at the proximal end of the elongate body.

53. The scope of claim 52, the tip cap further comprising one or more liquid stand offs such that, when the tip cap is coupled to the tip engagement region, the one or more liquid stand offs are configured to engage a portion of the tip face such that a liquid flow from the liquid outlet is directed towards the opening to further improve viewing through the visualization component.

54. The scope of any of claims 41-53, wherein the elongate body is rigid.

55. The scope of any of claims 41-53, wherein the elongate body is semi-rigid.

56. The scope of any of claims 41-53, wherein the elongate body is flexible.

57. The scope of claim 41 having an elongate body that is flexible or semi-rigid, the scope further comprising a handle including a steering mechanism for controlling a bend angle in the elongate body.

58. A surgical scope, comprising:
an elongate body having a proximal end and a distal end;
an interior lumen within the elongate body extending from the proximal end to the distal end;
a recessed portion at the elongate body distal end configured to releasably couple to a tip cap;
a tip face directly adjacent to the recessed portion and covering the interior lumen distal end;
two or more gas conduits within the elongate body lumen, each of said two or more gas conduits having an outlet in the tip face and an inlet at a gas manifold;
a gas inlet at the proximal end of the elongate body in communication with the gas manifold;
a visualization component in the tip face; and
a visualization component cable connected to the visualization component and in communication with the proximal end of the elongate body,
wherein an overall dimension of the recessed portion of the elongate body distal end is less than the overall dimension of the elongate body proximal portion.

59. The surgical scope of claim 58, further comprising a handle on the elongate body proximal end supporting the gas conduit inlet and the visualization component cable.

60. The surgical scope of claim 58, further comprising a liquid conduit within the elongate body lumen having an outlet in the tip face and an inlet at the proximal end of the elongate body.

61. The surgical scope of claim 60, further comprising a handle on the elongate body proximal end supporting the gas conduit inlet, the liquid conduit inlet and the visualization component cable.

62. The surgical scope of claim 59 or 60, wherein the gas manifold is disposed within the handle.

63. The scope of any of the above claims 58-62 wherein the elongate body is rigid, semi-rigid or flexible.

64. The scope of claim 63 having an elongate body that is flexible or semi-rigid, the handle further comprising: a steering mechanism for controlling a bend angle in a portion of the flexible or semi-rigid elongate body.

65. The scope of any of claims 27-64, wherein an angle formed by the tip face and the elongate body distal end is one of 90 degrees, 45 degrees and 30 degrees.

66. The scope of claim 65, wherein the tip cap is configured to couple to a tip face that forms an angle of 90 degrees, 45 degrees and 30 degrees.

67. The scope of any of the above claims 27-66, wherein the visualization component comprises a lens system.

68. The scope of claim 67, wherein the visualization component further comprises a solid state sensor, wherein the solid-state sensor is selected from the following group:
a Charge Coupled Device (CCD);
an Intensified Charge Coupled Device (ICCD);
an Electron Multiplying Charge Coupled Device (EMCCD); and
a Complementary Metal Oxide Semiconductor (CMOS) device.

69. A scope as in any of the above claims 27-68, wherein the visualization component is a part of a tip face of a sterilizable elongate body.

70. A scope as in any of the above claims 27-69, wherein the visualization component is a disposable part of a disposable tip cap.

71. A scope as in any of the above claims 27-70, wherein the visualization component comprises a lens system having a plurality of lens that together form an image with a field of view of between 60 and 140 degrees.

72. A scope as in any of the above claims 27-71, wherein the scope, tip face, tip cap and visualization device are configured for carrying out a procedure selected from the following group:
(a) a gastroscopy procedure by forming an image with a field of view of 120 to 140 degrees;
(b) an ERCP procedure by forming an image with a field of view of the camera head of the invention 120 to 140 degrees in a motherscope and by forming an image with a field of view of 100 degrees in a baby scope;
(c) a colonoscopy procedure by forming an image with a field of view of 120 to 140 degrees;
(d) a gynecology procedure by forming an image with a field of view of 100 to 120 degrees;
(e) a bronchoscopy procedure by forming an image with a field of view of 80 to 100 degrees;
(f) an ENT procedure by forming an image with a field of view of 80 to 100 degrees; and
(g) a transgastric procedure by forming an image with a field of view of 120 to 140 degrees in a motherscope and by forming an image with a field of view of 100 to 120 degrees in a baby scope.

73. A scope as in any of the above claims 27-72, wherein the visualization device includes a sensor having a diagonal size in the range from approximately 0.5 mm, 1 mm, 1.5 mm, 2 mm, 2.5 mm, 3 mm, or 3.5 mm, or 4 mm.

74. A scope, comprising:
an elongate body having a proximal end and a distal end and a non-round cross section;
a visualization component at the elongate body distal end; and
an attachment mechanism on the elongate body configured for attachment to a sheath such that, when a sheath is placed around the elongate body and attached thereto with the attachment mechanism, at least one conduit is configured to attach to an gas supply and extends from the proximal end to the distal end between an outer circumference of the elongate body and an inner circumference of the sheath.

75. The scope of claim 74, wherein the attachment mechanism is on a proximal portion of the elongate body and is configured for sealing engagement with the sheath.

76. The scope of claim 74, wherein the sheath includes a sidewall with an exterior wall having a circular cross section shape and an interior wall configured for complementary engagement with the non-round cross section of the elongate body.

77. The scope of claim 74, wherein the at least one conduit comprises a plurality of conduits.

78. The scope of claim 74, wherein the plurality of conduits are configured to direct gas over the visualization component in a vortex.

79. The scope of claim 77, wherein a fluid flow through the conduits is apportioned so as to adjust the flow characteristics of the fluid discharged from the plurality of conduits relative to the visualization component.

80. The scope of claim 74, wherein when the sheath is placed around the elongate body and attached thereto with the attachment mechanism, the at least one conduit is connected to a gas nozzle at the distal portion of the conduit, wherein the gas nozzle is configured to direct gas across the visualization component to provide at least one visual field improvement action.

81. The scope of claim 74, wherein when the sheath is placed around the elongate body and attached thereto with the attachment mechanism, one or more stand offs in a distal portion of the sheath engage with a portion of the elongate body distal end.

82. The scope of claim 74, wherein when the sheath is placed around the elongate body and attached thereto with the attachment mechanism, one or more stand offs in a distal portion of the sheath engage with a portion of the elongate body distal end and at least two conduits are formed along the elongate body in communication with a sheath gas inlet, and wherein a fluid flowing through the sheath gas inlet passes through the at least two conduits and exits adjacent to the visualization component via one or more openings bounded at least in part by a portion of one or more stand offs and a portion of the elongate body distal end.

83. The scope of claim 74, wherein when the sheath is placed around the elongate body and attached thereto with the attachment mechanism, a distal portion of the sheath having one or more stand offs engages a portion of the elongate body distal portion such that a gas flow introduced into the conduit is directed towards the visualization component.

84. The scope of claim 74, wherein when the sheath is placed around the elongate body and attached thereto with the attachment mechanism, a distal portion of the sheath having or more stand offs engage a portion of the elongate body distal portion such that a gas flow introduced into the conduit provides at least one visual field improvement action.

85. The scope of claim 74, wherein when the sheath is placed around the elongate body and attached thereto with the attachment mechanism, one or more stand offs in a distal portion of the sheath engage with a portion of the elongate body distal end and at least two conduits are formed along the elongate body in communication with a sheath gas inlet, and wherein a fluid flowing through the sheath gas inlet passes through the at least two conduits and exits via one or more openings bounded at least in part by a portion of one or more stand offs and a portion of the elongate body distal end, wherein the exiting gas flows provide at least one visual field improvement action for the visualization component.

86. The scope as in any of the above claims 74-85, the sheath further comprising one or more features configured to apportion gas between the at least two conduits.

87. The scope as in any of the above claims 74-85, the sheath further comprising one or more features distal to a sheath inlet to adjust the flow characteristics of the fluid discharged from the at least one conduit relative to the visualization component.

88. The scope as in any of claims 86-87 wherein the one or more features adjusts the relative velocity of the flow through the at least two conduits.

89. The scope of any of claims 74-88, wherein the at least one conduit comprises a first conduit and a second conduit, and wherein the first conduit is configured to have a first flow of gas and the second conduit is configured to have a second flow of gas, the first flow having a higher velocity than the second flow.

90. The scope of any of the above claims 74-89, further comprising a channel disposed completely within the sheath and in communication with an inlet at the sheath proximal end and having an outlet adjacent to the elongate body distal end.

91. The scope of claim 90, wherein the outlet is positioned adjacent to the exiting gas flows whereby the fluid provided via the outlet cooperates with the exiting gas flows to provide at least one visual field improvement action for the visualization component.

92. The scope of any of claims 80, 84, 85 and 91, wherein the visual field improvement action is one or more of: a gas flow pattern relative to the visualization component to remove condensation therefrom, a gas flow pattern relative to the visualization component to form an gas barrier to reduce or minimize particles in the visual field of the visualization component and a gas flow pattern relative to the visualization component to facilitate removal of a fluid applied to the visualization component.

93. The scope of claim 74 further comprising a visualization component cable connected to the visualization component.

94. The scope of any of the above claims 74-93, wherein the at least one attachment mechanism is configured to releasably couple with the sheath using one or more snap fit features.

95. The scope of any of the above claims 74-93, wherein the at least one attachment mechanism is configured to releasably couple with the sheath using a gas tight friction fit.

96. The scope of any of the above claims 74-93, wherein the at least one attachment feature is configured to releasably couple with the sheath and an o-ring in a compression fit.

97. The scope of any of the above claims 74-96, wherein the elongate body is rigid, semi-rigid or flexible.

98. The scope of claim 97 having an elongate body that is flexible or semi-rigid, further comprising a handle having a steering mechanism for controlling a bend angle in a portion of the flexible or semi-flexible elongate body.

99. The scope of any of the above claims 74-98, wherein the non-round cross section shape has a substantially circular perimeter with at least a portion of the perimeter having at least one flattened portion.

100. The scope of any of the above claims 74-98, wherein the non-round cross section shape has a substantially circular perimeter with at least a portion of the perimeter having at least one non-circular portion.

101. The scope of any of the above claims 74-98, wherein the non-round cross section shape has a substantially ovoid perimeter with at least a portion of the perimeter having at least one flattened portion.

102. The scope of any of the above claims 74-98, wherein the non-round cross section shape has a substantially ovoid perimeter with at least a portion of the perimeter having at least one non-ovoid portion.

103. The scope of any of the above claims 74-98, wherein the non-round cross section shape has a substantially elliptical perimeter with at least a portion of the perimeter having at least one flattened portion.

104. The scope of any of the above claims 74-98, wherein the non-round cross section shape has a substantially elliptical perimeter with at least a portion of the perimeter having at least one non-elliptical portion.

105. The scope of any of the above claims 74-98, wherein the non-round cross section shape has a substantially triangular perimeter.

106. The scope of any of the above claims 74-98, wherein the non-round cross section shape has a substantially triangular perimeter with at least a portion of each corner of the triangular perimeter having at least one flattened portion.

107. The scope of any of the above claims 74-98, wherein the non-round cross section shape has a substantially triangular perimeter and each of the corners are rounded.

108. The scope of any of the above claims 74-98, wherein the non-round cross section shape has a substantially triangular perimeter and each of the corners are rounded and at least two of the corners have about the same radius of curvature.

109. The scope of any of the above claims 74-98, wherein the non-round cross section shape has a substantially circular perimeter with at least one cut out portion.

110. The scope of any of the above claims 74-98, wherein the non-round cross section shape has a substantially circular perimeter with a plurality of cut outs along the perimeter.

111. The scope of any of the above claims 99-110, wherein the sheath has an exterior wall having a substantially circular cross section shape and an interior wall forming a lumen sized, shaped, adapted and configured for a complimentary fit with the elongate body non-round cross section shape.

112. A sheath for use with a non-round scope, comprising:
a tube having a proximal end and a distal end;
an interior wall of the tube defining an interior lumen extending from the proximal end to the distal end sized to receive the scope with the shape of the interior lumen selected for a complementary fit with the exterior shape of the non-round scope;
a gas inlet in the proximal end of the sheath;
a first portion of the interior wall having a first shape;
a second portion of the interior wall having a second shape;
wherein when the scope is positioned within the interior lumen, the interior wall of the tube and the exterior wall of the scope are positioned such that a first channel is formed by the first portion of the interior wall and a first portion of the exterior wall of the scope and a second channel is formed by the second portion of the interior wall and a second portion of the exterior wall of the scope such that a gas introduced in a proximal end of the first and second channels flows across a distal face of the non-round scope.

113. The sheath of claim 112 wherein the first gas conduit is in communication with a first gas outlet at the distal end of the sheath, and the second gas conduit is in communication with a second gas outlet at the distal end of the sheath.

114. The sheath of claim 112 or 113, further comprising a visualization component in the scope distal end and an opening in a distal portion of the sheath sized for use with the visualization component, the sheath having one or more stand offs wherein when the scope is positioned within the sheath the opening is appropriately positioned relative to the visualization component and the one or more stand offs are adapted and configured to engage a portion of the scope distal face whereby the gas flows from the first gas outlet and the second gas outlet are directed towards the opening to further at least one visual field improvement action.

115. The sheath of any of claims 112-114, wherein first channel is configured to have a first flow of gas and the second channel is configured to have a second flow of gas, the first flow having a higher velocity than the second flow.

116. The sheath of any of claims 112-115, further comprising a manifold in communication the gas inlet and with the first channel and the second channel.

117. The sheath as in any of claims 112-116, further comprising one or more features distal to the gas inlet wherein the flow into the sheath from the inlet is apportioned between the at least two conduits.

118. The sheath as in any of claims 112-117, further comprising one or more features distal to the gas inlet to adjust the flow characteristics of the fluid discharged from the first channel and the second channel relative to the visualization component.

119. The sheath as in any of claims 112-118, further comprising one or more features distal to the gas inlet to apportion the flow between the first conduit and the second conduit to adjust the flow characteristics of the gas flow relative to the visualization component.

120. The sheath of any of claims 112-119, wherein the one or more features adjusts the relative velocity of the flow through the first channel and the second channel.

121. The sheath of any of claims 112-120, wherein the exiting gas flows from the first channel and the second channel provide at least one visual field improvement action for the visualization component.

122. The sheath of claim 121, wherein the visual field improvement action is one or more of: a gas flow pattern relative to the visualization component to remove condensation therefrom, a gas flow pattern relative to the visualization component to form an gas barrier to reduce or minimize particles in the visual field of the visualization component and a gas flow pattern relative to the visualization component to facilitate removal of a fluid applied to the visualization component.

123. The sheath of claim 121, further comprising one or more liquid stand offs positioned within the distal portion of the sheath wherein when the sheath is coupled to the scope, the one or more liquid stand offs engage a portion of the distal portion of the scope whereby a liquid flow from the liquid outlet is directed towards the opening to further at least one visual field improvement action.

124. The sheath of any of claims 112-123, further comprising a liquid conduit within the sheath or formed as a third conduit between the sheath and the scope having a liquid outlet in relation to the scope distal end and an inlet at the sheath proximal end.

125. The sheath of any of the claims 112-124, further comprising a channel disposed completely within the sheath and in communication with an inlet at the sheath proximal end and having an outlet adjacent to the scope distal end.

126. The sheath of claim 125, wherein the outlet is positioned adjacent to the exiting gas flows whereby the fluid provided via the outlet cooperates with the exiting gas flows to provide at least one visual field improvement action for the visualization component.

127. The sheath of claims 123 and 123, wherein the visual field improvement action is one or more of: a gas flow pattern relative to the visualization component to remove condensation therefrom, a gas flow pattern relative to the visualization component to form an gas barrier to reduce or minimize particles in the visual field of the visualization component and a gas flow pattern relative to the visualization component to facilitate removal of a fluid applied to the visualization component.

128. The sheath of any of claims 112-127, wherein the sheath is adapted and configured for cooperative operation with an scope having an elongate body that is rigid, semi-rigid or flexible.

129. The sheath of claim 128, further comprising a handle coupled to the scope having a steering mechanism or a bending mechanism for controlling a bend angle in a portion of the flexible or semi-flexible elongate body of the scope.

130. The sheath of any of claims 112-129, further comprising at least one attachment feature adapted and configured using one or more snap fit features, a gas tight friction fit or an o-ring in a compression fit to releasably couple the sheath with the non-round scope inserted into the sheath.

131. The sheath of any of claims 112-130, wherein the non-round scope has an elongate body that is rigid, semi-rigid or flexible.

132. The sheath of claim 131, the non-round scope having an elongate body that is flexible or semi-rigid, further comprising a handle for use with the sheath and non-round scope combination having a bending or steering mechanism for controlling a bend angle in a portion of the flexible or semi-rigid elongate body.

133. The sheath or scope in any of the above claims 74-132, being adapted and configured for use with a visualization component positioned within an scope distal end that is one of 90 degrees, 45 degrees and 30 degrees.

134. The sheath or scope of any of the above claims 74-133, wherein the visualization component comprises a lens system.

135. The sheath or scope of claim 134, wherein the visualization component further comprises a solid state sensor, wherein the solid-state sensor is selected from the following group:
a Charge Coupled Device (CCD);
an Intensified Charge Coupled Device (ICCD);
an Electron Multiplying Charge Coupled Device (EMCCD); and
a Complementary Metal Oxide Semiconductor (CMOS) device.

136. The sheath or scope of any of the above claims 74-135, wherein the visualization component is a part of a tip face of a sterilizable elongate body of a non-round scope.

137. The sheath or scope of any of the above claims 74-135, wherein the visualization component of a non-round scope comprises a lens system having a plurality of lens that together form an image with a field of view of between 60 and 140 degrees.

138. The sheath or scope of any of the above claims 74-136, wherein the non-round scope, the sheath and the visualization component are adapted and configured for carrying out a procedure selected from the following group:
(a) a gastroscopy procedure by forming an image with a field of view of 120 to 140 degrees;
(b) an ERCP procedure by forming an image with a field of view of the camera head of the invention 120 to 140 degrees in a motherscope and by forming an image with a field of view of 100 degrees in a baby scope;
(e) a colonoscopy procedure by forming an image with a field of view of 120 to 140 degrees;
(d) a gynecology procedure by forming an image with a field of view of 100 to 120 degrees;
(e) a bronchoscopy procedure by forming an image with a field of view of 80 to 100 degrees;
(f) an ENT procedure by forming an image with a field of view of 80 to 100 degrees; and
(g) a transgastric procedure by forming an image with a field of view of 120 to 140 degrees in the motherscope and by forming an image with a field of view of 100 to 120 degrees in the baby scope.

139. The sheath or scope of any of the above claims 74-138, wherein the visualization component includes a sensor as in claim 93 having a diagonal size in the range from approximately 0.5 mm, 1 mm, 1.5 mm, 2 mm, 2.5 mm, 3 mm, or 3.5 mm, or 4 mm.

140. The sheath of claim 112, wherein the first and second channels are together configured to direct gas over the lens in a vortex.

141. A method of using the scope of any of the above claims, comprising:
inserting the scope into a human or animal body during a procedure;
visualizing a portion of the body using a visualization component of the scope; and
operating a view optimizing assembly to perform at least one visual improvement action.

142. The method of claim 141, wherein the visual field improvement action is one or more of: a gas flow pattern relative to the visualization component to remove condensation therefrom, a gas flow pattern relative to the visualization component to form an gas barrier to reduce or minimize particles in the visual field of the visualization component and a gas flow pattern relative to the visualization component to facilitate removal of a fluid applied to the visualization component.

143. The method of claim 141, wherein the visual improvement action is performed without removing the scope from the human or animal body during the procedure.

144. The method of claim 141, further comprising steering the scope by bending or orienting a flexible section of the scope, wherein the visual improvement action continues during the steering step.

145. The method of claim 141, further comprising supplying gas to the view optimizing assembly from a gas supply.

146. The method of claim 145, wherein the gas supply is an insufflator.

147. The method of claim 141, wherein a portion of the human or animal body is insufflated during the procedure.
